(19) United States
(12) Patent Application Publication
(10) Pub. No.: US 2016/0368836 A1
(43) Pub. Date: Dec. 22, 2016

(54) PROCESS FOR THE AROMATIZATION OF A METHANE-CONTAINING GAS STREAM USING ZIRCONIUM HYDROGEN ACCEPTOR PARTICLES

(71) Applicant: SHELL OIL COMPANY, Houston, TX (US)

(72) Inventors: Peter Tanev TANEV, Katy, TX (US); Daniel Edward GERWIEN, Sugar Land, TX (US); Shaojun MIAO, Katy, TX (US); Anthony Tyler SIMPSON, Katy, TX (US); Elizabeth Colquhoun HOWLAND, Houston, TX (US); Steven Sangyun LIM, Kingwood, TX (US)

(21) Appl. No.: 15/182,954

(22) Filed: Jun. 15, 2016

Related U.S. Application Data
(60) Provisional application No. 62/180,864, filed on Jun. 17, 2015.
(60) Provisional application No. 62/216,686, filed on Sep. 10, 2015.

(51) Int. Cl. C07C 2/76; B01J 29/90; B01J 38/10; B01J 29/48

(52) U.S. Cl. CPC C07C 2/76; B01J 29/48; B01J 29/90; B01J 38/10; C07C 2529/48

(57) ABSTRACT

Implementations of the disclosed subject matter provide a process for the aromatization of a methane-containing gas stream that includes contacting the methane-containing gas stream in a reaction zone of an aromatization reactor comprising an aromatization catalyst and a zirconium hydrogen acceptor under methane-containing gas aromatization conditions to produce a product stream comprising aromatics and hydrogen, wherein at least a portion of the produced hydrogen is bound by the zirconium hydrogen acceptor in the reaction zone and removed from the product and the reaction zone as zirconium hydride, and wherein the weight ratio of zirconium hydrogen acceptor to the aromatization catalyst is at least 1:1.

### CROSS-REFERENCE TO RELATED APPLICATIONS

This application claims priority to U.S. Provisional Application Ser. No. 62/180,864 filed Jun. 17, 2015, the entire disclosure of which is hereby incorporated by reference. This application also claims priority to U.S. Provisional Application Ser. No. 62/216,686 filed Sep. 10, 2015, the entire disclosure of which is hereby incorporated by reference. This application is also related to co-pending U.S. patent application Ser. No. 14/395,819, entitled “AROMATIZATION OF A METHANE-CONTAINING GAS STREAM”, which claims priority to U.S. Provisional Application No. 61/636,915 filed on Apr. 23, 2012, the disclosure of which is incorporated herein by reference. This application is also related to co-pending U.S. patent application Ser. No. 14/395,821, entitled “A PROCESS FOR THE AROMATIZATION OF A METHANE-CONTAINING GAS STREAM”, which claims priority to U.S. Provisional Application No. 61/636,906 filed on Apr. 23, 2012, the disclosure of which is incorporated herein by reference.

### TECHNICAL FIELD OF THE INVENTION

This invention relates to a process for the aromatization of a methane-containing gas stream in a reactor containing both catalyst and zirconium hydrogen acceptor particles, wherein the zirconium hydrogen acceptor particles bind the produced hydrogen insitu from the methane aromatization reaction thereby shifting the thermodynamic equilibrium of the reaction and resulting in a significantly higher CH₄ conversion and aromatics yields than the maximum allowable by the equilibrium.

### BACKGROUND

The aromatic hydrocarbons (specifically benzene, toluene and xylenes) are the main high-octane bearing components of the gasoline pool and important petrochemical building blocks used to produce high value chemicals and a variety of consumer products, for example, styrene, phenol, polymers, plastics, medicines, and others. Since the late 1930's, aromatics are primarily produced by upgrading of oil-derived feedstocks via catalytic reforming or cracking of heavy naphthas. However, occasional severe oil shortages and oil price spikes result in severe aromatics shortages and aromatics price spikes. Therefore, there is a need to develop new, independent from oil, commercial routes to produce high value aromatics from highly abundant and inexpensive hydrocarbon feedstocks such as methane or stranded natural gas (which typically contains about 80-90% vol. methane).

There are enormous proven reserves of stranded natural gas around the world. According to some estimates, the world reserves of natural gas are at least equal to those of oil. However, unlike the oil reserves that are primarily concentrated in a few oil-rich countries and are extensively utilized, upgraded and monetized, the natural gas reserves are much more broadly distributed around the world and significantly underutilized. Many developing countries that have significant natural gas reserves lack the proper infrastructure to exploit them and convert or upgrade them to higher value products. Quite often, in such situations, natural gas is flared to the atmosphere and wasted. Because of the above reasons, there is enormous economic incentive to develop new technologies that can efficiently convert methane or natural gas to higher value chemical products, specifically aromatics.

In 1993, Wang et al., (Catal. Lett. 1993, 21, 35-41), discovered a direct, non-oxidative route to partially convert methane to benzene by contacting methane with a catalyst containing 2.0% wt. Molybdenum on an H-ZSM-5 zeolite support at atmospheric pressure and a temperature of 700° C. Since Wang's discovery, numerous academic and industrial research groups have become active in this area and have contributed to further developing various aspects of the direct, non-oxidative methane to benzene catalyst and process technology. Many catalyst formulations have been prepared and tested and various reactor and process conditions and schemes have been explored.

Despite these efforts, a direct, non-oxidative methane aromatization catalyst and process cannot yet be commercialized. Some important challenges that need to be overcome to commercialize this process include: (i) the very low, as dictated by thermodynamic equilibrium, per pass methane conversion and benzene yield (for example, 10% wt. and 6% wt., respectively at 700° C.); (ii) the fact that the reaction is favored by high temperature and low pressure; (iii) the need to separate the produced aromatics and hydrogen from unreacted (mainly methane) hydrocarbon off gas and (iv) the rapid coke formation and deposition on the catalyst surface and corresponding relatively fast catalyst deactivation. Among these challenges, overcoming the thermodynamic equilibrium limitations and significantly improving (e.g., by greater than 3 times) the methane conversion and benzene yield per pass has the potential to enable the commercialization of an efficient, direct, non-oxidative methane-containing gas aromatization process.

The methane aromatization reaction can be described for the particular case of methane to benzene as follows:

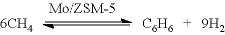

According to the reaction, 6 molecules of methane are required to generate a molecule of benzene. It is also apparent that, the production of a molecule of benzene is accompanied by the production of 9 molecules of hydrogen. Simple thermodynamic calculations revealed and experimental data have confirmed that, the methane aromatization at atmospheric pressure is equilibrium limited to about 10 or 20% wt. at reaction temperatures of 700° C. or 800° C., respectively. In addition, experimental data showed that the above conversion levels correspond to about 6 and 11.5% wt. benzene yield at 700° C. and 800° C., respectively. The aforementioned low per pass methane conversions and benzene yields are not attractive enough to provide an economic justification for scale-up and commercialization of a methane-containing gas aromatization process.

Therefore, there is a need to develop an improved direct, non-oxidative methane aromatization process that provides for significantly higher (than those allowed by the thermodynamic equilibrium) methane conversion and benzene yields per pass.

### BRIEF SUMMARY

The invention provides a process for the aromatization of a methane-containing gas stream comprising: contacting the methane-containing gas stream in a reaction zone of a reactor comprising an aromatization catalyst and a zirconium hydrogen acceptor under methane-containing gas aromatization conditions to produce a product stream comprising aromatics and hydrogen wherein at least a portion of the produced hydrogen is bound by the zirconium hydrogen acceptor in the reaction zone and removed from the product stream and the reaction zone, and wherein the weight ratio of zirconium hydrogen acceptor to the aromatization catalyst is at least 1:1.

The invention further provides a novel process and reactor schemes that employ single or multiple catalysts and/or zirconium hydrogen acceptor beds.

The invention also provides several catalyst and/or zirconium hydrogen acceptor recycle and regeneration process schemes. According to these schemes, the catalyst and/or t zirconium hydrogen acceptor particles are regenerated simultaneously or separately in single or in separate vessels and then returned to the reactor for continuous (uninterrupted) production of aromatics and hydrogen. The aforementioned insitu hydrogen removal in the reaction zone allows for overcoming the thermodynamic equilibrium limitations of the methane aromatization reaction and for shifting the reaction equilibrium to the right. This results in significantly higher and economically more attractive methane-containing gas stream conversion and benzene yields per pass relative to the case without hydrogen removal, i.e. without zirconium hydrogen acceptor in the reaction zone. Additional features, advantages, and embodiments of the disclosed subject matter may be set forth or apparent from consideration of the following detailed description, drawings, and claims. Moreover, it is to be understood that both the foregoing summary and the following detailed description are examples and are intended to provide further explanation without limiting the scope of the claims.

### BRIEF DESCRIPTION OF THE DRAWINGS

The accompanying drawings, which are included to provide a further understanding of the disclosed subject matter, are incorporated in and constitute a part of this specification. The drawings also illustrate embodiments of the disclosed subject matter and together with the detailed description serve to explain the principles of embodiments of the disclosed subject matter. No attempt is made to show structural details in more detail than may be necessary for a fundamental understanding of the disclosed subject matter and various ways in which it may be practiced.

FIG. 1 shows a schematic diagram of an embodiment of the presently disclosed subject matter: a fixed-bed aromatization reactor with catalyst and zirconium hydrogen particles intermixed in a fixed bed or stationary configuration.

FIG. 2 shows a schematic diagram of an embodiment of the presently disclosed subject matter: regeneration of the intermixed catalyst and zirconium hydrogen acceptor particles in a single regeneration vessel.

FIG. 3 shows a schematic diagram of another embodiment of the presently disclosed subject matter: separation and regeneration of catalyst and zirconium hydrogen acceptor particles in separate vessels followed by mixing of both types of particles before feeding them back to reactor.

FIG. 4 shows the relationship between methane conversion and time on stream (hereinafter denoted as “TOS”) based on various embodiments of the presently disclosed subject matter and comparative examples.

FIG. 5 shows the relationship between benzene yield and time on stream based on various embodiments of the presently disclosed subject matter and comparative examples.

FIG. 6 shows the relationship between naphthalene yield and time on stream based on various embodiments of the presently disclosed subject matter and comparative examples.

FIG. 7 shows the relationship between hydrogen yield and time on stream based on various embodiments of the presently disclosed subject matter and comparative examples.

FIG. 8 shows the powder XRD patterns obtained for fresh (as-received) and spent (i.e., saturated with hydrogen) zirconium acceptor particles based on the methane aromatization process of an embodiment of the presently disclosed subject matter.

FIG. 9 shows the relationship between methane conversion, operating pressure and time on stream based on various embodiments of the presently disclosed subject matter and comparative examples.

FIG. 10 shows the relationship between benzene yield, operating pressure and time on stream based on various embodiments of the presently disclosed subject matter and comparative examples.

FIG. 11 shows the relationship between methane conversion, zirconium acceptor and catalyst particles size and zirconium acceptor/catalyst particles weight ratio based on various embodiments of the presently disclosed subject matter.

FIG. 12 shows the relationship between benzene conversion, zirconium acceptor and catalyst particles size and zirconium acceptor/catalyst particles weight ratio based on various embodiments of the presently disclosed subject matter.

FIG. 13 shows the relationship between methane conversion and time on stream based on various embodiments of the presently disclosed subject matter.

FIG. 14 shows the relationship between benzene yield and time on stream based on various embodiments of the presently disclosed subject matter.

FIG. 15 shows the relationship between hydrogen yield and time on stream based on various embodiments of the presently disclosed subject matter.

### DETAILED DESCRIPTION

The conversion of a methane-containing gas stream to aromatics is typically carried out in a reactor comprising a solid catalyst substance, which is active in the conversion of the methane-containing gas stream to aromatics. The methane-containing gas stream that is fed to the reactor comprises more than 50% vol. methane, preferably more than 70% vol. methane and more preferably of from 75% vol. to 100% vol. methane. The balance of the methane-containing gas may be other low molecular weight alkanes, for example, ethane, propane and butane. The methane-containing gas stream may be natural gas which is a naturally occurring hydrocarbon gas mixture consisting primarily of methane, with up to about 30% vol. concentration of other hydrocarbons (usually mainly ethane and propane), as well as small amounts of other impurities such as carbon dioxide, nitrogen and others. The aromatization reaction of this invention is carried out in a reactor, for example, a fixed bed reactor. To enable this, suitably shaped and sufficiently robust catalyst and zirconium hydrogen acceptor particles that are able to sustain the rigors of high severity reactor operation are prepared and used for the reaction.

According to the present invention, the use of specific operating conditions for the aromatization process and particular weight ratios of zirconium hydrogen acceptor particles to catalyst particles in the reaction zone provides several advantages over the prior art. The present invention provides an efficient zirconium H₂ acceptor and preferred operating conditions for the aromatization of methane-containing gas stream consisting of contacting the methane-containing gas stream in a reactor comprising methane aromatization catalyst particles and zirconium hydrogen acceptor particles, where the weight ratio of zirconium hydrogen acceptor particles to catalyst particles (Zr:Catalyst) is at least 1:1. The hydrogen acceptor material used in this reaction is a zirconium comprising particulate material that has the ability, when subjected to aromatization operating conditions, to selectively accept or react with hydrogen to form a sufficiently strong hydrogen-zirconium acceptor bond. The zirconium hydrogen acceptor reversibly binds the hydrogen in such a way that during operation in the reactor, the hydrogen is strongly bound to the zirconium hydrogen acceptor under the methane containing gas aromatization conditions. In addition, the zirconium hydrogen acceptor is able to release the hydrogen when subjected to regeneration conditions that favor release of the previously bound hydrogen and regeneration of the zirconium hydrogen acceptor. The present invention provides an efficient, high temperature zirconium hydrogen acceptor material that is capable of shifting the thermodynamic equilibrium of the methane aromatization reaction to significantly (e.g., greater than 3 times at 700° C.) higher than the maximum allowable CH₄ conversion and benzene yields.

The conversion of a methane-containing gas stream is carried out at particular operating conditions which lead to improved conversion and benzene yields. For example, the process of the present invention may be carried out at a gas hourly space velocity of from 100 to 40,000 h⁻¹, a pressure of from 0.5 to 10 bara and a temperature of from 500 to 900° C. More preferably, the conversion is carried out at a gas hourly space velocity of from 300 to 30,000 h⁻¹, a pressure of from 0.5 to 5 bara and a temperature of from 600 to 800° C. Even more preferably, the conversion is carried out at a gas hourly space velocity of from 500 to 10,000h⁻¹, a pressure of from 0.5 to 3 bara and a temperature of from 650 to 750° C. In an embodiment, the pressure may be at least 2 bara, and according to an embodiment, the pressure may be at least 3 bara. The methane aromatization reaction is carried out until conversion falls to values that are lower than those that are economically acceptable. At this point, the aromatization catalyst has to be regenerated to restore its aromatization activity to a level similar to its original activity. The regeneration of the catalyst could be carried out separately from the zirconium hydrogen acceptor or in the presence of the zirconium hydrogen acceptor. Also the regeneration of the zirconium hydrogen acceptor could be carried out separately from the catalyst or in the presence of the catalyst. Following the regeneration, the catalyst is again contacted with the zirconium hydrogen acceptor and a methane-containing gas stream in the reaction zone of the aromatization reactor for continuous production of aromatics.

Any catalyst suitable for methane-containing gas aromatization can be used in the process of this invention. The catalyst typically comprises one or more active metals on an inorganic oxide support and optionally comprises promoters and other beneficial compounds. The active metal or metals, promoters, compounds and the inorganic support all contribute to the overall aromatization activity, mechanical strength and performance of the aromatization catalyst.

The active metal component(s) of the catalyst may be any metal that exhibits catalytic activity when contacted with a methane-containing gas stream under methane aromatization conditions. The active metal may be selected from the group consisting of: vanadium, chromium, manganese, zinc, iron, cobalt, nickel, copper, gallium, germanium, niobium, molybdenum, ruthenium, rhodium, silver, tantalum, tungsten, rhenium, platinum and lead and mixtures thereof. The active metal is preferably molybdenum.

The promoter or promoters may be any element or elements that, when added in a certain preferred amount and by a certain preferred method during catalyst synthesis, improve the performance of the catalyst in the methane aromatization reaction.

The inorganic oxide support can be any support that, when combined with the active metal or metals and optionally the promoter or promoters contributes to the overall catalyst performance exhibited in the methane aromatization reaction. The support has to be suitable for treating or impregnating with the active metal compound or solution thereof and a promoter compound or solution thereof. The inorganic support preferably has a well-developed porous structure with a sufficiently high surface area and pore volume and suitable for aromatization surface acidity. The inorganic oxide support may be selected from the group consisting of zeolites, non-zeolitic molecular sieves, silica, alumina, zirconia, titania, yttria, ceria, rare earth metal oxides and mixtures thereof. The inorganic oxide support of this invention preferably contains zeolite as the primary component. The zeolite is selected from the group consisting of ZSM-5, ZSM-22, ZSM-8, ZSM-11, ZSM-12 or ZSM-35 zeolite structure types. The zeolite is preferably a ZSM-5 zeolite. The ZSM-5 zeolite further may have a SiO₂/Al₂O₃ ratio of 10 to 100. Preferably, the SiO₂/Al₂O₃ ratio of the zeolite is in the range of 20-50. Even more preferably the SiO₂/Al₂O₃ ratio is from 20 to 40 and most preferably from about 20 to 30. The support may optionally contain about 5-70% wt of a binder that binds the zeolite powder particles together and allows for shaping of the catalyst in the desired form and for achieving the desired high catalyst mechanical strength. More preferably the support contains from 15-30% wt. binder. The binder is selected from the group consisting of silica, alumina, zirconia, titania, yttria, ceria, lanthana, and other rare earth oxides or mixtures thereof.

The final shaped catalyst could be in the form of cylindrical pellets, rings or spheres. The preferred catalyst shape of this invention is spherical or cylindrical pellets. The spherical or pelletized catalyst of this invention could be prepared by any method known to those skilled in the art. Preferably, the spherical catalyst of this invention is prepared via spray drying of zeolite containing sols of appropriate concentration and composition. The zeolite containing sol may optionally contain binder. The spherical catalyst has a particle size distribution and predominant particle size or diameter that makes it suitable for use in the disclosed process. The spherical particle diameter of the catalyst of this invention is preferably selected to be in the range of 20 microns to 3 mm More preferably, the spherical catalyst of this invention has particle diameter in the range of 50 microns to 2 mm. As an example, particle size may be based on the prevalent particle size as determined from a particle size distribution. For example, if a particle size distribution is measured (e.g., using for example, the light scattering method) of the spherical catalyst particles, the prevalent particle size may appear as a peak in the particle size distribution plot of the number of particles versus particle size. The cylindrical pelletized catalyst of this invention is prepared by extrusion of suitable extrusion mix containing appropriate concentrations of zeolite powder and optionally binder. The diameter of the cylindrical catalyst pellets is selected to be in the range of from 1 to 4 mm.

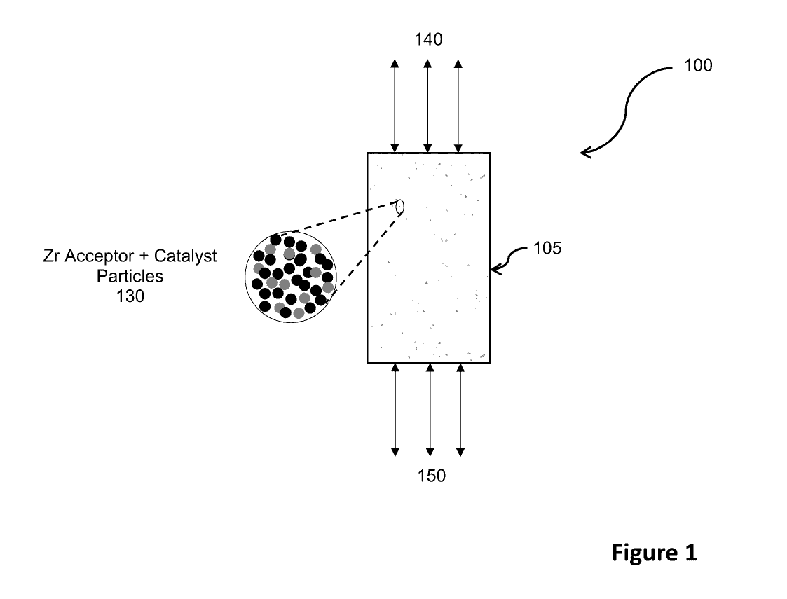

In addition to the particular process conditions of the present invention described above, by combining a specific weight ratio of at least 1:1 zirconium hydrogen acceptor particles to catalyst particles (i.e., Zr:Catalyst) in the reaction zone, significantly higher CH₄ conversion and benzene yields can be achieved by the process of the present invention. A feature of the methane aromatization process of this invention is that it provides for insitu removal of the hydrogen product from the reaction zone and significant thermodynamic equilibrium shift by use of a zirconium hydrogen acceptor particles combined with catalyst particles in the reaction zone. As a result, a significant advantage of the disclosed subject matter is that it provides for a substantial increase in both methane conversion and benzene yield per pass. This results in methane conversion and benzene yield values that are significantly higher (e.g., greater than 3 times at 700° C.) relative to those achieved for the same methane aromatization reaction and operating conditions but without the use of zirconium hydrogen acceptor. The presently disclosed subject matter is enabled by mixing and combining specific zirconium hydrogen acceptor particles and catalyst particles amounts to achieve a weight ratio, for example of at least 1:1, of the zirconium hydrogen acceptor particles to catalyst particles in the reaction zone or the aromatization reactor (see FIGS. 1-3) under the particular process conditions described herein. The weight ratio of zirconium hydrogen acceptor particles to the aromatization catalyst particles may be from about at least 1:1, about 1:1 to 10:1, about 2:1 to 4:1, at least 4:1, and at least 6:1. The shaped zirconium hydrogen acceptor particles may be in the form of irregular particles, cylindrical pellets, rings, tablets or spheres. The preferred zirconium hydrogen acceptor particle shapes are pellets, rings or spheres. The preferred particle size of the zirconium hydrogen acceptor of this invention is preferably selected to be in the range of 50 micron to 2 mm.

The usage of zirconium hydrogen acceptor particles in a reactor when operating under methane aromatization conditions provides for the quick removal of the produced hydrogen from the reaction zone and for shifting the methane aromatization reaction equilibrium toward greater methane conversion and benzene yield per pass. The zirconium hydrogen acceptor used in this reaction can be a zirconium metal, zirconium-comprising alloy or a zirconium-comprising compound that, when subjected to aromatization operating conditions, selectively accepts, absorbs or reacts with hydrogen to form a sufficiently strong zirconium-hydrogen bond (such as for example in zirconium hydride). The zirconium hydrogen acceptor reversibly binds the hydrogen in such a way that during operation in the reactor the hydrogen is strongly bound to the zirconium acceptor under the methane-containing gas stream aromatization conditions. In addition, the zirconium hydrogen acceptor is preferably able to release the hydrogen when transported to the regeneration section where it is subjected to a different set of (regeneration) conditions that favor release of the previously bound hydrogen and regeneration of the zirconium hydrogen acceptor. Additionally, the zirconium hydrogen acceptor may include one or more other metals. For example, the zirconium hydrogen acceptor may include one or more metals that may enhance the ability of the zirconium hydrogen acceptor to accept and or release hydrogen or improve the physical properties of the zirconium hydrogen acceptor to lead to, for example, greater metal phase stability, surface area, mechanical rigidity and others. According to the process of the present invention, the obtained conversion of the methane-containing gas stream is at least 35 wt %, and at least 50 wt %. The methane conversion in weight percent is calculated on the basis of experimental/test data as explained in the Examples section below. Furthermore, according to the process of the present invention, the obtained benzene yield per pass is at least 18 wt %, at least 20 wt %, and at least 25%. The benzene yield (adjusted for coke) is calculated also as explained in section below.

As an example, FIG. 1 shows an aromatization reactor with catalyst and zirconium hydrogen acceptor particles intermixed in a solids bed configuration. As shown, a reactor 100 with a solids bed 105 comprises a mixture 130 of catalyst and zirconium hydrogen acceptor particles. The process gas may flow downward into the solids bed 105 through gas inlet 140 and outward from the solids bed 105 through gas outlet 150, as shown by the arrows 140 and 150. Alternatively, the process gas may flow upwards (for example, as in the case of fluidized-bed reactor) using 150 as a gas inlet and 140 as a gas outlet.

Another advantage of the present invention is that, the particle shapes, sizes and mass of both zirconium hydrogen acceptor and catalyst particles can be designed and selected in such a way so that they can be combined and mixed well together in the reactor volume. In addition, they could be designed in such a way so that to provide for easy separation of particles by type following the reaction and prior to regeneration in separate vessels. Also, the invention provides for two or more different hydrogen acceptors (e.g., different based on chemical formula and/or physical properties) to be simultaneously used with the catalyst in the reactor bed to achieve the desired degree of hydrogen separation from the methane aromatization reaction zone.

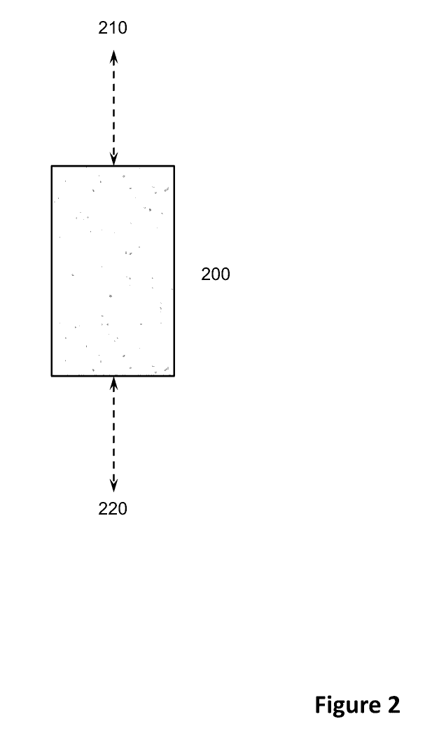

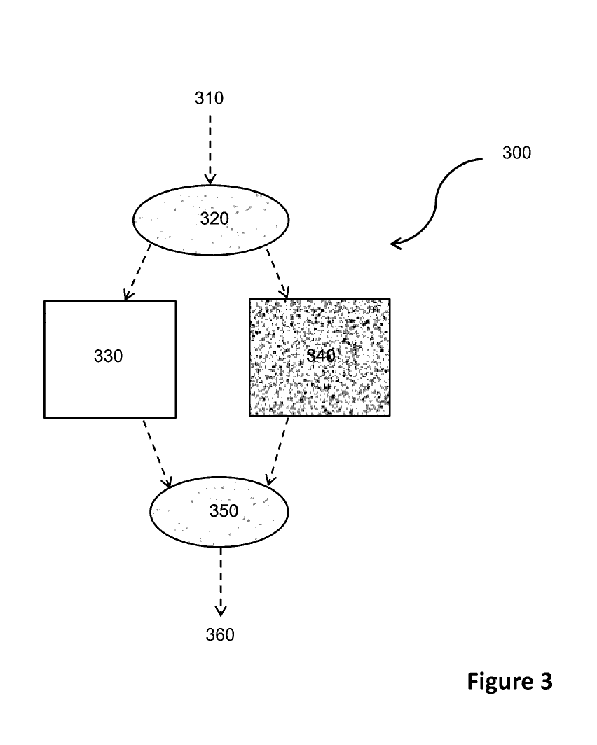

Another advantage of the process of this invention is that it provides for the catalyst and the zirconium hydrogen acceptor particles to be simultaneously regenerated in the reactor (e.g., as shown in FIG. 1) or withdrawn from the reaction zone, regenerated in a separate vessel or vessels according to one of the schemes illustrated in FIGS. 2 and 3 and then returned to the reactor for aromatics and hydrogen production. In addition, a method of regenerating the zirconium hydrogen acceptor and reusing it in the methane aromatization reaction to afford performance very similar to the one of the fresh zirconium acceptor is also provided. The zirconium hydrogen acceptor and catalyst particles regeneration can be accomplished either simultaneously or stepwise in the reactor illustrated in FIG. 1 or in a different regeneration vessel as illustrated in FIG. 2 or regenerated separately in separate vessels as illustrated in FIG. 3. The later operation schemes (e.g., as shown in FIGS. 2 and 3) provide for maximum flexibility to accomplish the hydrogen release or regeneration of the zirconium hydrogen acceptor particles and catalyst particles under different and suitable for the purpose operating conditions. The regeneration of the zirconium hydrogen acceptor and catalyst particles can be accomplished in fixed, moving or fluidized bed reactor vessels schematically shown in FIGS. 1-3. In the specific case of separate regeneration as illustrated in FIG. 3, the zirconium hydrogen acceptor particles can be separated from the catalyst on the basis of (but not limited to) differences in mass, particle size or density between the zirconium acceptor and the catalyst particles.

FIG. 2 shows a regenerator vessel 200 that is used to regenerate the zirconium hydrogen acceptor particles and regenerate the catalyst particles. The zirconium hydrogen acceptor and catalyst particles are introduced via inlet 210 and are then removed following the regeneration via outlet 220. During the regeneration, the regeneration gas could be fed downward in the direction from 210 to 220 or upward in the direction from 220 to 210. The hydrogen removed from the zirconium hydrogen acceptor during the regeneration and the gases produced during catalyst regeneration are removed from the regenerator via 210 or 220 or if needed, via additional outlets (not shown).

In FIG. 3, regenerator system 300 comprises a separation step 320 to separate the zirconium hydrogen acceptor particles from the catalyst particles. First, a mixture of spent zirconium acceptor and catalyst particles is fed from the methane aromatization reactor via line 310. Following the separation in 320, the catalyst particles are fed to the catalyst regeneration vessel 330, and the zirconium hydrogen acceptor particles are fed to zirconium hydrogen acceptor regeneration vessel 340. The regenerated catalyst particles and zirconium hydrogen acceptor particles are then mixed back together in mixing step 350 and then fed back to the methane aromatization reactor via line 360. This regeneration scheme is also suitable for the aromatization reactor shown on FIG. 1.

The methane aromatization catalyst forms coke during the reaction. An accumulation of coke on the surface of the catalyst gradually covers the active for methane aromatization sites of the catalyst resulting in gradual reduction of its aromatization activity. Therefore, the coked catalyst has to be regenerated at a certain carefully chosen frequency insitu in the reactor as illustrated in FIG. 1 or removed from the reaction zone of the aromatization reactor and regenerated in one of the regeneration vessel(s) as illustrated in FIGS. 2 and 3. The regeneration of the catalyst could be carried out by any of the methods known to those skilled in the art while the zirconium hydrogen acceptor particles are completely withdrawn or still within the reaction zone of the aromatization reactor.

The regeneration of the catalyst can be carried out by any method known to those skilled in the art. For example, two possible regeneration methods are hot hydrogen stripping and oxidative burn at temperatures sufficient to remove the coke from the surface of the catalyst. If hot hydrogen stripping is used to regenerate the catalyst, then at least a portion of the hydrogen used for the catalyst regeneration may come from the hydrogen released from the zirconium hydrogen acceptor. Additionally, fresh hydrogen may be fed to the catalyst regeneration vessel as needed to properly supplement the hydrogen released from zirconium hydrogen acceptor and to complete the catalyst regeneration. If the catalyst and zirconium acceptor regeneration is carried out in the same vessel (see FIGS. 1-2), then the hydrogen removed from the zirconium hydrogen acceptor insitu or exsitu can at least partially hydrogen strip and regenerate the catalyst.

If the regeneration of catalyst and zirconium hydrogen acceptor particles is carried out in different vessels, the operating conditions of each vessel can be optimized, selected and maintained to favor the regeneration of the catalyst particles or the zirconium hydrogen acceptor particles, respectively. Hydrogen removed from the zirconium hydrogen acceptor particles can be used to at least partially hydrogen strip and regenerate the catalyst particles.

Yet another advantage of the process of this invention is that it provides for the release of the hydrogen that is bound to the zirconium hydrogen acceptor when the saturated acceptor is subjected to the regeneration conditions in the regeneration vessel(s). Furthermore, the released hydrogen can be utilized to regenerate the catalyst particles, or it may be subjected to any other suitable chemical use, or monetized to improve the overall aromatization process economics.

Another advantage of the present invention is that it allows for different regeneration conditions to be used in the different regeneration vessels to optimize and minimize the regeneration time required for the catalyst and zirconium hydrogen acceptor particles and to improve their performance in the methane aromatization reaction.

### Examples

In fixed bed methane aromatization performance tests carried out under different operating conditions, and with different Zr:Catalyst weight ratios, it was discovered that the operating conditions, homogeneity of the Zr acceptor and catalyst particles mixing, and Zr:Catalyst particles weight ratio have a profound effect on the degree of methane aromatization thermodynamic equilibrium shift (i.e. on the degree of increase of the CH₄ conversion and corresponding aromatics and hydrogen product yields beyond those dictated by the equilibrium). In addition, it was discovered that the Zr acceptor particle size does have an effect on the degree of equilibrium shift, i.e. on the CH₄ conversion and aromatics yields. Finally, it was determined that at optimal Zr:Catalyst particles weight ratio (for example, 4:1), the increase of the operating pressure from 1 to 3 bara does not have an adverse effect on the CH₄ conversion and benzene yield.

I. Materials:

Zirconium Hydrogen Acceptor:

Pure zirconium metal granule-shaped particles (made by American Elements, 1-2 mm granule size, PN# ZR-M-0251M-GR.1T2MM) were used as a zirconium hydrogen acceptor material. Prior to use, the zirconium metal particles were stored under inert gas (argon) atmosphere in order to prevent the formation of zirconium oxide. In order to obtain smaller size Zr acceptor particles and to study the effect of Zr particle size on methane aromatization performance, the above large Zr particle size granules were ground and sieved to obtain 10 times smaller Zr particle size fraction of 0.1-0.2 mm.

M2B Catalyst:

An H-ZSM-5 zeolite powder (Zeolyst, ID# CBV3024) was pressed, crushed, and sieved to obtain a particle fraction of size in the range of 0.1-0.2 mm or fraction of the size in the range of 1-2 mm. The zeolite particles were then dried under a flow of dry air at 125° C. for 1 hour, and subsequently calcined using a 3° C./min heating rate to 500° C. and by holding at this temperature for 4 hours. A 200 grams quantity of the so calcined zeolite particles fraction were then impregnated with 160 mL of an aqueous solution of Mo(C₂O₄)₃ to afford an 8 wt % loading of Mo. The resulting impregnated material was then dried under a flow of dry air at 100° C. for 2 hours. Following the drying, the catalyst was calcined again in a flow of dry air by using a 3° C./min heating rate to 300° C. and holding at this temperature for 2 hours and then heated using a 3° C./min heating rate to 500° C. and holding at this temperature for 3 hours. The obtained methane aromatization catalyst was found to contain 8% wt Mo/H-ZSM-5.

II. Catalyst Pretreatment and Reactor Loading Protocols:

Catalyst Pretreatment/Reduction:

Prior to activity tests, a quantity of 10 cc (approximately 6.5 g) of the dry methane aromatization catalyst described above were placed in a quartz reactor, purged with inert gas and then reduced insitu at 1 bara with a 20 L/hr (GHSV=2000 h⁻¹) flow of pure hydrogen. The temperature profile used for the catalyst reduction was as follows: 0.5° C./min heating rate to 240° C. and hold for 5 hours, 2.0° C./min heating rate to 480° C. and hold for 2 hours, 2.0° C./min heating rate to 700° C. and hold for 1.5 hours. The pre-reduced methane aromatization catalyst was then cooled in hydrogen to 400° C., then cooled to ambient temperature under 20 L/hr of argon (GHSV=2000 h⁻¹) and kept sealed in the reactor under argon “blanket”.

Catalyst/Reactor Loading:

Following the reduction of the catalyst, specific amounts of the zirconium hydrogen acceptor particles were selected/measured so as to achieve Zr acceptor:catalyst (Zr:Cat) particles weight ratio in the range of 1:1 to 6:1. The so measured amount of Zr acceptor particles were then mixed with 6.5-6.6 g of pre-reduced catalyst under inert atmosphere, and loaded into a quartz reactor for testing. The loading of the zirconium acceptor was accomplished while purging the (reduced) catalyst bed with argon at a sufficiently high flow rate to fluidize the catalyst bed and to allow for good intermixing of the methane aromatization catalyst and the zirconium hydrogen acceptor particles. The zirconium acceptor particles were then slowly dropped from above into the fluidized catalyst bed in order to allow for homogeneous intermixing of acceptor and catalyst particles. This is accomplished by gradually adding/dropping small portions of acceptor particles into the reactor with the fluidized catalyst and gradually reducing the argon gas flow. Following the loading, the argon flow was stopped, and the reactor inlet and outlet immediately blocked to maintain an inert environment within the reactor. The reactor, with the well-mixed zirconium hydrogen acceptor particles and methane aromatization catalyst particles was then placed into a reactor furnace and connected to gas supply, outlet and GC sampling lines in preparation for testing.

III Activity Testing:

Activity tests were carried out in the above described quartz fixed-bed reactor. The selected test conditions were as follows:

| | | |
|---|---|---|
| Temperature | 700° | C. |
| Pressure | 1-3 | bara |
| Feed gas composition | 100 | v % CH₄ |
| GHSV | 1000 | h⁻¹ |
| Catalyst amount | 10 | cc |

(approximately
6.5 or 6.6 g
depending on the
Zr and catalyst
particle size)

The GHSV values in the following examples (when Zr acceptor particles were present) were calculated solely on the basis of the catalyst volume. The catalytic performance data were gathered by taking GC sample shots at 10-minute intervals via a fully-automated GC-sampling system. The CH₄ conversion, benzene, naphthalene, and hydrogen yields were used as criteria for the evaluation of methane aromatization activity.

A. Zr-to-Catalyst Weight Ratio of 1:1, GHSV=1000 h⁻¹, Zr and Catalyst Particles Size=1-2 mm, Pressure=1 bara

This sample was prepared and loaded in the reactor as described above, by mixing 6.5 g of zirconium hydrogen acceptor particles and 10 cc (approximately 6.5 g) of pretreated methane aromatization catalyst particles to obtain a 1:1 zirconium hydrogen acceptor particles/methane aromatization catalyst particles weight ratio. The sample was then tested as described above, using a methane flow rate of 10 L/hr (GHSV=1000 h⁻¹) and operating pressure of 1 bara. The sample was found to exhibit maximum CH₄ conversions of 20 wt %, corresponding to maximum benzene yield of 9 wt % and maximum naphthalene yield of 7 wt %. At the point of maximum CH₄ conversion, the H₂ yield was only 2.6 wt %.

B. Zr-to-Catalyst Weight Ratio of 1:1, GHSV=1000 h⁻¹, Zr and Catalyst Particles Size=0.1-0.2 mm, Pressure=1 bara

This sample was prepared and loaded in the reactor as described above, by mixing 6.6 g of zirconium hydrogen acceptor particles and 10 cc (approximately 6.6 g) of methane aromatization catalyst particles to obtain a 1:1 zirconium hydrogen acceptor/methane aromatization catalyst weight ratio. The sample was then tested as described above, using a methane flow rate of 10 L/hr (GHSV=1000 h-1) and operating pressure of 1 bara. The maximum CH₄ conversion attained by this sample was 20.5 wt %, corresponding to a maximum benzene yield of 10.4 wt % and a maximum naphthalene yield of 7.3 wt %.

C. Zr-to-Catalyst Weight Ratio of 2:1, GHSV=1000 h⁻¹, Zr and Catalyst Particles Size=1-2 mm, Pressure=1 bara

This sample was prepared and loaded in the reactor as described above, by mixing 13 g of zirconium hydrogen acceptor particles and 10 cc (approximately 6.5 g) of pretreated methane aromatization catalyst particles to obtain a 2:1 zirconium hydrogen acceptor/methane aromatization catalyst weight ratio. The sample was then tested as described above, using a methane flow rate of 10 L/hr (GHSV=1000 h⁻¹) and operating pressure of 1 bara. The sample was found to exhibit maximum CH₄ conversions of 42.8 wt %, a corresponding maximum benzene yield of 16 wt % and maximum naphthalene yield of 22.2 wt %. At the point of maximum CH₄ conversion, the H₂ yield was only approximately 1.4 wt %.

D. Zr-to-Catalyst Weight Ratio of 2:1, GHSV=1000 h⁻¹, Zr and Catalyst Particles Size=0.1-0.2 mm, Pressure=1 bara

This sample was prepared and loaded in the reactor as described above, by mixing 13.2 g of zirconium hydrogen acceptor particles and 10 cc (approximately 6.6 g) of methane aromatization catalyst particles to obtain a 2:1 zirconium hydrogen acceptor/methane aromatization catalyst weight ratio. The sample was then tested as described above, using a methane flow rate of 10 L/hr (GHSV=1000 h-1) and operating pressure of 1 bara. The maximum CH₄ conversion attained by this sample was 40.7 wt %, and the corresponding maximum benzene yield was 22.3 wt % and the maximum naphthalene yield was 13.7 wt %.

E. Zr-to-Catalyst Weight Ratio of 4:1, GHSV=1000 h⁻¹, Zr and Catalyst Particles Size=1-2 mm, Pressure=1 bara

This sample was prepared and loaded in the reactor as described above, by mixing 26 g of zirconium hydrogen acceptor particles and 10 cc (approximately 6.5 g) of pretreated methane aromatization catalyst particles to obtain a 4:1 zirconium hydrogen acceptor particles/methane aromatization catalyst particles weight ratio. The sample was then tested as described above, using a methane flow rate of 10 L/hr (GHSV=1000 h⁻¹) and operating pressure of 1 bara. The sample was found to exhibit maximum CH₄ conversions of 60 wt %, corresponding to maximum benzene yield of 33.2 wt % and maximum naphthalene yield of 23.2 wt %. At the point of maximum CH₄ conversion, the H₂ yield was only approximately 0.6 wt %. This represents about 80% lower hydrogen yield compared to the hydrogen yield exhibited by the reference catalyst alone, i.e. in the absence of the zirconium hydrogen acceptor (see Comparative Example Zr-to-Catalyst Weight Ratio of 0:1 below).

F. Zr-to-Catalyst Weight Ratio of 4:1, GHSV=1000 h⁻¹, Zr and Catalyst Particles Size=0.1-0.2 mm, Pressure=1 bara

This sample was prepared and loaded in the reactor as described above, by mixing 26.4 g of zirconium hydrogen acceptor particles and 10 cc (approximately 6.6 g) of methane aromatization catalyst particles to obtain a 4:1 zirconium hydrogen acceptor particles/methane aromatization catalyst particles weight ratio. The sample was then tested as described above, using a methane flow rate of 10 L/hr (GHSV=1000 h⁻¹) and operating pressure of 1 bara. The maximum CH₄ conversion attained by this sample was 50.5 wt %, corresponding to a maximum benzene yield of 30.2 wt % and a maximum naphthalene yield of 19.4 wt %.

G. Zr-to-Catalyst Weight Ratio of 6:1, GHSV=1000 h⁻¹, Zr and Catalyst Particles Size=1-2 mm, Pressure=1 bara

This sample was prepared and loaded in the reactor as described above, by mixing 39 g of zirconium hydrogen acceptor particles and 10 cc (approximately 6.5 g) of methane aromatization catalyst particles to obtain a 6:1 zirconium hydrogen acceptor particles/methane aromatization catalyst particles weight ratio. The sample was then tested as described above, using a methane flow rate of 10 L/hr (GHSV=1000 h-1) and operating pressure of 1 bara. The maximum CH₄ conversion attained by this sample was 68.7 wt %, corresponding to a maximum benzene yield of 34.8 wt % and a maximum naphthalene yield of 41.1 wt %. At the point of maximum CH₄ conversion, the corresponding H₂ yield was only about 0.28 wt %. This represents greater than 80% reduction of the hydrogen yield due to the hydrogen absorption by the Zr hydrogen acceptor compared to the hydrogen yield observed for the reference catalyst alone, i.e. in the absence of hydrogen acceptor (see Comparative Example Zr-to-Catalyst Weight Ratio of 0:1 below).

H. Zr-to-Catalyst Weight Ratio of 6:1, GHSV=1000 h⁻¹, Zr and Catalyst Particles Size=0.1-0.2 mm, Pressure=1 bara

This sample was prepared and loaded in the reactor as described above, by mixing 39.6 g of zirconium hydrogen acceptor particles and 10 cc (approximately 6.6 g) of methane aromatization catalyst particles to obtain a 6:1 zirconium hydrogen acceptor particles/methane aromatization catalyst particles weight ratio. The sample was then tested as described above, using a methane flow rate of 10 L/hr (GHSV=1000 h⁻¹) and operating pressure of 1 bara. The maximum CH₄ conversion attained by this sample was 73.9 wt %, corresponding to a maximum benzene yield of 37.4 wt % and a maximum naphthalene yield of 35.6 wt %.

I. Zr-to-Catalyst Weight Ratio of 4:1, GHSV=1000 h⁻¹, Zr and Catalyst Particles Size=1-2 mm, Pressure=2 bara

This sample was prepared and loaded in the reactor as described above, by mixing 26 g of zirconium hydrogen acceptor particles and 10 cc (approximately 6.5 g) of methane aromatization catalyst particles to obtain a 4:1 zirconium hydrogen acceptor particles/methane aromatization catalyst particles weight ratio. The sample was then tested as described above, using a methane flow rate of 10 L/hr (GHSV=1000 h⁻¹) and operating pressure of 2 bara. The maximum CH₄ conversion exhibited by this sample was 73.9 wt %, corresponding to maximum benzene yield of 33.1 wt % and a maximum naphthalene yield of 36.2 wt %.

J. Zr-to-Catalyst Weight Ratio of 4:1, GHSV=1000 h⁻¹, Zr and Catalyst Particles Size=1-2 mm, Pressure=3 bara

This sample was prepared and loaded in the reactor as described above, by mixing 26 g of zirconium hydrogen acceptor particles and 10 cc (approximately 6.5 g) of methane aromatization catalyst particles to obtain a 4:1 zirconium hydrogen acceptor particles/methane aromatization catalyst particles weight ratio. The sample was then tested as described above, using a methane flow rate of 10 L/hr (GHSV=1000 h⁻¹) and operating pressure of 3 bara. The maximum CH₄ conversion attained by this sample was 61.3 wt %, corresponding to a maximum benzene yield of 30.3 wt % and a maximum naphthalene yield of 25.9 wt %.

K. Zr-to-Catalyst Weight Ratio of 1:0, GHSV=1000 h⁻¹, Zr and Catalyst Particles Size=1-2 mm, Pressure=1 bara

This sample is representative of the prior art. The sample was prepared and loaded into the reactor as described above, by using only 6.5 g (1-2 mm) of zirconium hydrogen acceptor particles and omitting the methane aromatization catalyst particles from the reactor loading to obtain a 1:0 zirconium hydrogen acceptor particles/methane aromatization catalyst particles weight ratio. No hydrogen pretreatment was performed on the zirconium hydrogen acceptor particles. The sample was then tested under the test conditions described above, using a methane flow rate of 10 L/hr (GHSV=1000 h⁻¹) and operating pressure of 1 bara. This sample was found to be completely inactive for methane aromatization, i.e., no CH₄ conversion or benzene, naphthalene, or H₂ yields were observed throughout the test.

L. Zr-to-Catalyst Weight Ratio of 1:0, GHSV=1000 h-1, Zr and Catalyst Particles Size=0.1-0.2 mm, Pressure=1 bara

This sample is also representative of the prior art. The sample was prepared and loaded into the reactor as described above, by using only 6.6 g of zirconium acceptor particles and by omitting the methane aromatization catalyst particles from the reactor loading to obtain a 1:0 zirconium hydrogen acceptor particles/methane aromatization catalyst particles weight ratio. No hydrogen pretreatment was performed on the zirconium hydrogen acceptor particles. The sample was then tested as under the test conditions described above, using a methane flow rate of 10 L/hr (GHSV=1000 h⁻¹) and operating pressure of 1 bara. This sample was found to be completely inactive for methane aromatization, i.e., no CH₄ conversion or benzene, naphthalene, or H₂ yields were observed throughout the test.

M. Zr-to-Catalyst Weight Ratio of 0:1, GHSV=1000 h⁻¹, Zr and Catalyst Particles Size=1-2 mm, Pressure=1 bara

This sample is representative of the prior art. The sample was prepared and loaded in the reactor as described above, using 10 cc (approximately 6.5 g) of methane aromatization catalyst particles and omitting the zirconium hydrogen acceptor particles from the reactor to obtain a 0:1 zirconium hydrogen acceptor particles/methane aromatization catalyst particles weight ratio. The sample was then tested as described under the test conditions described above, using a methane flow rate of 10 L/hr (GHSV=1000 h⁻¹) and operating pressure of 1 bara. The catalytic performance in methane aromatization of this sample was found to be exactly as expected—close to the one that is the maximum allowed by the methane to benzene thermodynamic equilibrium limitations. Specifically, the maximum CH₄ conversion exhibited by this sample was found to be approximately 11 wt %, benzene yield of 5.3 wt % and a naphthalene yield of 3.2 wt %. At the point of maximum CH₄ conversion, the hydrogen yield was found to be approximately 2.6 wt %.

N. Zr-to-Catalyst Weight Ratio of 0:1, GHSV=1000 h⁻¹, Zr and Catalyst Particles Size=1-2 mm, Pressure=2 bara

This sample is representative of the prior art. The sample was prepared and loaded in the reactor as described above, using 10 cc (approximately 6.5 g) of methane aromatization catalyst particles and omitting the zirconium hydrogen acceptor particles from the reactor to obtain a 0:1 zirconium hydrogen acceptor particles/methane aromatization catalyst particles weight ratio. The sample was then tested as described under the test conditions described above, using a methane flow rate of 10 L/hr (GHSV=1000h⁻¹) and operating pressure of 2 bara. The catalytic performance in methane aromatization of this sample was found to be as expected—close to the one that is the maximum allowed by the methane aromatization thermodynamic equilibrium limitations at 2 bara. Specifically, the maximum CH₄ conversion exhibited by this sample was found to be approximately 5 wt %, benzene yield of 2.5 wt % and a naphthalene yield of ˜1.5 wt %.

O. Zr-to-Catalyst Weight Ratio of 0:1, GHSV=1000 h⁻¹, Zr and Catalyst Particles Size=0.1-0.2 mm, Pressure=1 bara

This sample is representative of the prior art. The sample was prepared and loaded in the reactor as described above, using 10 cc (approximately 6.6 g) of methane aromatization catalyst particles and omitting the zirconium hydrogen acceptor particles from the reactor to obtain a 0:1 zirconium hydrogen acceptor particles/methane aromatization catalyst particles weight ratio. The sample was then tested as described under the test conditions described above, using a methane flow rate of 10 L/hr (GHSV=1000h⁻¹) and operating pressure of 1 bara. The catalytic performance of this sample was found to be exactly as expected—close to the one that is the maximum allowed by the methane aromatization thermodynamic equilibrium limitations at 1 bara. Specifically, the maximum CH₄ conversion exhibited by this sample was found to be approximately 11 wt %, benzene yield of 5 wt % and a naphthalene yield of 3 wt %. At the point of maximum CH₄ conversion, the hydrogen yield was found to be approximately 2.5 wt %.

P. Regeneration of Spent Zirconium Hydrogen Acceptor Particles:

Following the activity testing, the spent zirconium acceptor/catalyst particles mixture was cooled to room temperature under a flow of 20 L/hr of Argon (GHSV=2000 h⁻¹) and removed from the quartz reactor. The spent zirconium acceptor particles were then separated from the spent catalyst particles by offloading the reactor and by placing the zirconium acceptor/catalyst particles mixture in a short curved (at one end) quartz tube equipped with a coarse frit at the bottom (inlet side) of the quartz tube. On the top/outlet curved end the quartz tube was equipped with a tightly fit porous plastic bag which is used to collect the lighter catalyst particles. The light catalyst particles escaped at certain critical inert gas flow/fluidization rate from the quartz tube into the bag. Nitrogen was flowed through the tube at a sufficiently high flow rate so as to fluidize the zirconium acceptor:catalyst particles mixture. Due to the lower density/weight of the spent catalyst particles relative to the spent zirconium acceptor particles, the catalyst particles floated towards the top of the bed during the fluidization. The flow rate of the nitrogen was then gradually increased until a critical fluidization rate was achieved and the catalyst particles moved up through the top curve of the quartz tube and escaped out of the tube into the perforated plastic bag. The few catalyst particles remaining behind and among the zirconium acceptor particles were then removed manually with tweezers from the zirconium acceptor particles.

Next, the separated spent zirconium hydrogen acceptor particles were placed in a clean quartz reactor tube and purged with 20 L/hr flow of argon (GHSV=2000 h⁻¹) and subsequently heated from ambient temperature to 750-850° C. in 2 hours followed by a 2-hour hold at the final temperature. During this step, the spent zirconium acceptor particles released the hydrogen absorbed during the aromatization activity testing, returning in their original non-hydride/metallic state. The zirconium hydrogen acceptor particles were then cooled to ambient temperature under argon flow, the argon flow to the reactor was stopped, and the reactor inlet and outlet were immediately blocked to maintain an inert (under argon) environment within the reactor/around the regenerated zirconium acceptor particles. The regenerated zirconium acceptor particles were then stored under argon “blanket” in the blocked reactor until activity testing.

Q. Regenerated Zr Acceptor, Zr-to-Catalyst Weight Ratio of 4:1, GHSV=1000 h⁻¹, Zr and Catalyst Particles Size=1-2 mm, Pressure=1 bara

This sample was prepared and loaded in the reactor as described above, by mixing 26 g of the regenerated zirconium hydrogen acceptor particles as described above and 10 cc (approximately 6.5 g) of fresh methane aromatization catalyst particles to obtain a 4:1 zirconium hydrogen acceptor particles/methane aromatization catalyst particles weight ratio. Fresh methane aromatization catalyst particles rather than spent methane aromatization catalyst particles were used for this test in order to remove the effect of incompletely regenerated catalyst and to properly assess the ability/extent of zirconium hydrogen acceptor particles regeneration. The sample was then tested according to the test conditions described above, using a methane feed flow rate of 10 L/hr (GHSV=1000 h⁻¹) and operating pressure of 1 bara.

IV. Analytics

The catalytic performance data were gathered by taking GC sample shots of the full product from the aromatization reactor at approximately 10-minute intervals via a fully-automated GC-sampling system. The GC sampling system fed the GC sample shots into a custom designed GC analytical system consisting of two GC's working in parallel to analyze/speciate the full product composition. Basis the product composition, the CH₄ conversion, benzene, naphthalene and H₂ yields were calculated as described in paragraph V. below. The CH₄ conversion, benzene, naphthalene, and H₂ yields were used as criteria for the evaluation of methane aromatization activity/performance.

V. Results

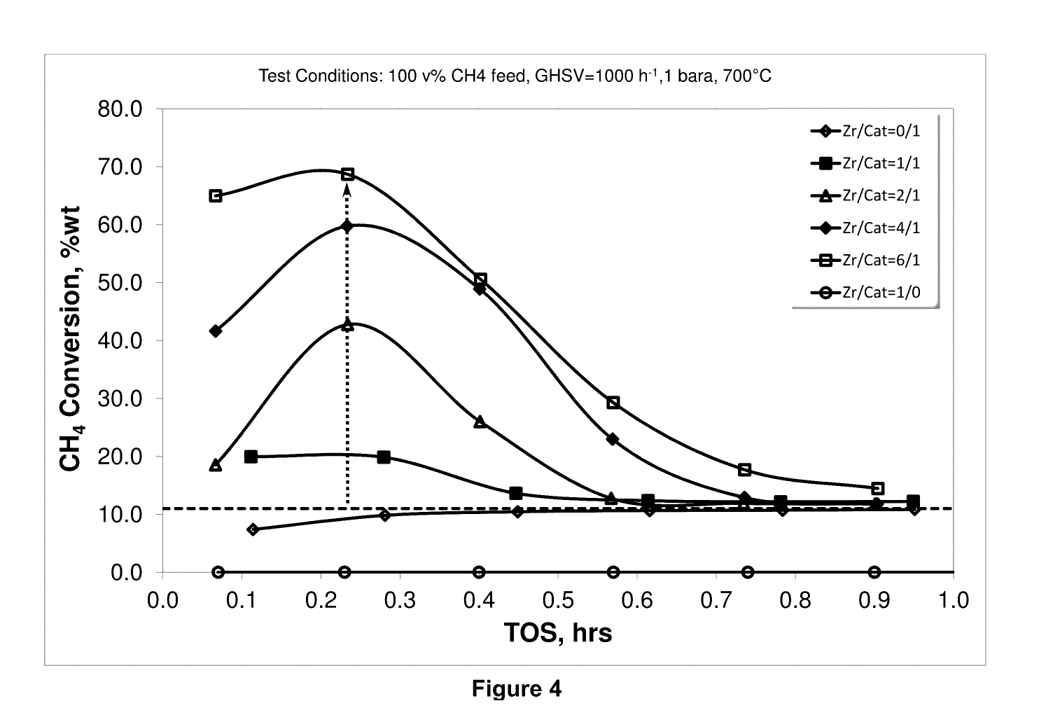

FIG. 4 shows the methane conversion vs. time on stream (TOS) data obtained according to the process of the present invention. In particular, FIG. 4 shows the CH₄ conversion vs. time on stream data obtained for different zirconium hydrogen acceptor/methane aromatization catalyst particles weight ratios. The predominant particle size for both, the zirconium acceptor particles and the catalyst particles was intentionally chosen to be similar, i.e. in the same range of particle sizes of from 1-2 mm. The test data were obtained using 100% vol CH₄ feed, GHSV=1000 h⁻¹, 1 bara and 700° C. The GHSV is a measure of the volume of gas passing through the volume of the catalyst per unit of time and is obtained by dividing the gas flow rate through the reactor expressed in cubic centimeter per hour (cc/hr) by the catalyst volume also expressed in cubic centimeters. As mentioned above, according to the process of the present invention, the obtained CH₄ conversion is at least 35 wt %, and at least 50 wt %. The CH₄ conversion in weight percent was calculated on the basis of experimental/test data by subtracting the CH₄ mass flow rate at the reactor outlet from the CH₄ mass flow rate at the reactor inlet and then dividing by the CH₄ mass flow rate at the reactor inlet and multiplying by 100, as shown below:

$${CH}_{4} Conversion , wt \% = \frac{[ {CH}_{4} Mass Flow Rate In - {CH}_{4} Mass Flow Rate out ]}{ {CH}_{4} Mass Flow Rate In} \times 100$$

The benzene yield (adjusted for coke) is calculated as the benzene mass produced (at the reactor outlet) per unit of time divided by the total (including coke on catalyst) mass flow from the reactor outlet, as shown below:

$$Benzene yield , wt \% = \frac{Benzene Mas s Out Per Unit of Time}{ Total Adjusted Mass ( Including Coke ) Flow Rate Out} \times 100$$

The naphthalene yield (adjusted for coke) is calculated as the naphthalene mass produced (at the reactor outlet) per unit of time divided by the total (including coke on catalyst) mass flow from the reactor outlet, as shown below:

$$Naphthalene yield , wt \% = \frac{Naphthalene Mas s Out Per Unit of Time}{ Total Adjusted Mass ( Including Coke ) Flow Rate Out} \times 100$$

The hydrogen yield (adjusted for coke) is calculated as the hydrogen mass produced (at the reactor outlet) per unit of time divided by the total (including coke on catalyst) mass flow from the reactor outlet, as shown below:

$$Hydrogen out , wt \% = \frac{Hydrogen Mas s Out Per Unit of Time}{ Total Adjusted Mass ( Including Coke ) Flow Rate Out} \times 100$$

The analysis of the data in FIG. 4 shows that in the absence of zirconium acceptor, the lined-out methane aromatization catalyst affords approximately 11% wt of methane conversion (see curve denoted with Ti/Cat=0/1 and open-diamond shaped line markers). This experimentally obtained methane conversion value matches the maximum allowed by the methane to benzene thermodynamic equilibrium methane conversion value at 1 bara and 700° C. On the other hand, the data obtained for the zirconium acceptor alone, i.e. without the methane aromatization catalyst (see curve denoted with Zr/Cat=1/0 and open circle shaped line markers) show that the zirconium acceptor alone does not exhibit methane conversion activity. Therefore, the zirconium acceptor alone is inactive, i.e. not capable to activate the methane aromatization reaction. In contrast, the data for the zirconium hydrogen acceptor and methane aromatization catalyst mixtures of the presently disclosed subject matter show that increasing the amount of zirconium hydrogen acceptor particles in the zirconium hydrogen acceptor particles and catalyst particles mixture (from a weight ratio of 1:1 to 6:1) leads to a very significant increase in the methane conversion to values significantly higher/beyond the value dictated by the thermodynamic equilibrium. Specifically, the mixture with 4:1 Zr:Catalyst particles weight ratio (see curve denoted with Zr/Cat=4/1 and solid diamond line markers) afforded approximately 60% wt methane conversion. In addition, the mixture with the highest Zr:Catalyst particles weight ratio of 6:1 afforded at the same conditions, even higher-methane conversion of approximately 70% wt (see curve denoted with Zr/Cat=6/1 and open square shaped line markers). This represents approximately seven times higher methane conversion relative to the maximum allowed conversion by the methane to benzene thermodynamic equilibrium at 1 bara and 700° C. This also represents about seven times higher methane conversion relative to the one typically obtained at the same set of operating conditions with the catalyst alone (i.e., without hydrogen acceptor according to the prior art).

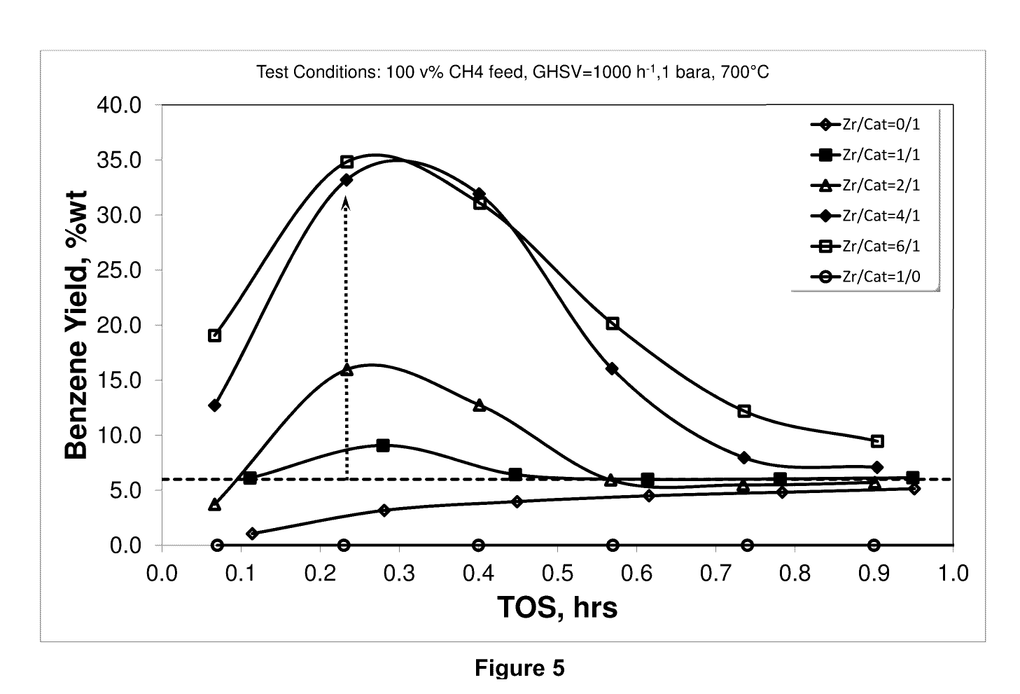

FIG. 5 shows the corresponding benzene yield vs. time on stream data obtained for the above samples. Due to the thermodynamic equilibrium limitations and the short duration (1 hr) of the test, the methane aromatization catalyst alone, i.e. in the absence of zirconium acceptor (see curve denoted with Zr/Cat=0/1 and open-diamond shaped line markers), afforded less than 6% wt benzene yield. Also, in accord with its lack of CH₄ conversion activity, no benzene yield/production was observed for the zirconium acceptor alone (see curve denoted with Zr/Cat=1/0 and open circle shaped line markers). In contrast, the data for the zirconium hydrogen acceptor and methane aromatization catalyst particles mixtures of this invention show that, increasing the amount of zirconium hydrogen acceptor particles in the zirconium acceptor and methane aromatization catalyst particles mixtures (from weight ratios of 1:1 to 6:1) leads to a very significant increase in the benzene yield beyond the one dictated by the thermodynamic equilibrium. More specifically, the zirconium acceptor: catalyst particles mixture with weight ratio of 4:1 (see curve denoted with Zr/Cat=4/1 and solid diamond shaped line markers) afforded approximately 33% wt. benzene yield. In addition, the mixture with the highest Zr:Catalyst particles weight ratio of 6:1 afforded somewhat higher, approximately 35 wt % benzene yield (see curve denoted with Zr/Cat=6/1 and open square shaped line markers). Therefore, in accord with the magnitude of the methane conversion advantage, the benzene yield advantage afforded by the mixture of the present invention comprising 6:1 zirconium acceptor and methane aromatization catalyst particles weight ratio is also about six times higher benzene yield than the one observed for the methane aromatization catalyst alone. Furthermore, according to the process of the present invention, the obtained benzene yield per pass is at least 33 wt %, and at least 35 wt %, as shown in FIG. 5. These significantly higher benzene yields (e.g., at least six times higher), relative to the ones dictated by the methane aromatization thermodynamic equilibrium and observed/reported in the prior art, undoubtedly makes the commercialization of the zirconium hydrogen acceptor assisted methane aromatization process of this invention a more attractive, than the prior art equilibrium limited methane aromatization processes, from an economics stand point proposition.

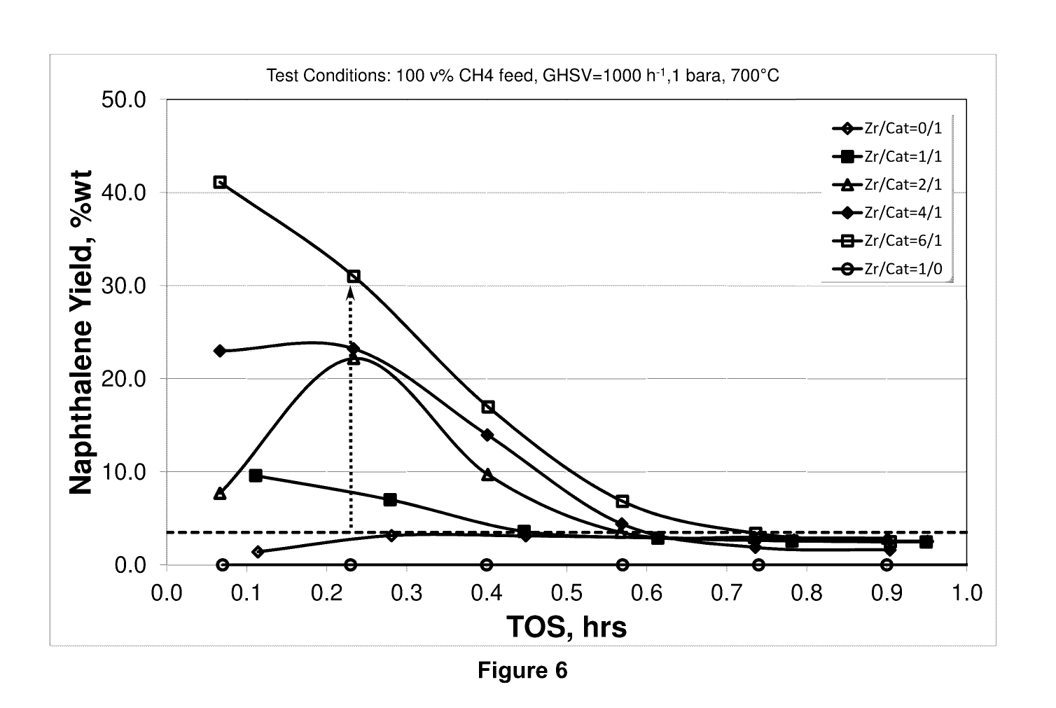

FIG. 6 shows the corresponding naphthalene yields vs time on stream data obtained for the above samples. The naphthalene yield afforded by the methane aromatization catalyst alone (see curve denoted with Zr/Cat=0/1 and open-diamond shaped line markers) was found to be very small, i.e. approximately 3% wt. On the other hand, since the zirconium acceptor alone (see curve denoted with Zr/Cat=1/0 and open circle shaped line markers) is not active for methane aromatization, it did not afford any appreciable yield of naphthalene. In contrast, the data for the zirconium acceptor and methane aromatization catalyst particles mixtures of this invention show again that, increasing the amount of zirconium hydrogen acceptor particles in the zirconium acceptor and methane aromatization catalyst particles mixtures of the presently disclosed subject matter (from weight ratios of 1:1 to 6:1) leads to a very significant increase in the naphthalene yields beyond those expected by thermodynamic equilibrium. Specifically, the mixture with zirconium acceptor to methane aromatization catalyst particles weight ratios of 4:1 afforded, naphthalene yield of about 23 wt % (see curve denoted with Zr/Cat=4/1 and solid diamond line markers). Furthermore, the mixture with Zr:Catalyst particles weight ratio of 6:1 afforded approximately 31 wt % naphthalene yield (see curve denoted with Zr/Cat=6/1 and open-square shaped line markers). This is a very significant (e.g., approximately up to ten times higher) increase in the naphthalene yield beyond the one dictated by the thermodynamic equilibrium limitations (usually about 3% wt). The significantly higher yields of naphthalene, relative to the ones allowed by equilibrium/afforded by the prior art, makes the commercialization of a zirconium hydrogen acceptor assisted methane aromatization process of the present invention a more attractive proposition from an economics stand point. It should be noted that even higher CH₄ conversion and aromatics yields may be possible at higher weight ratios of zirconium acceptor to catalyst particles in the aromatization reactor.

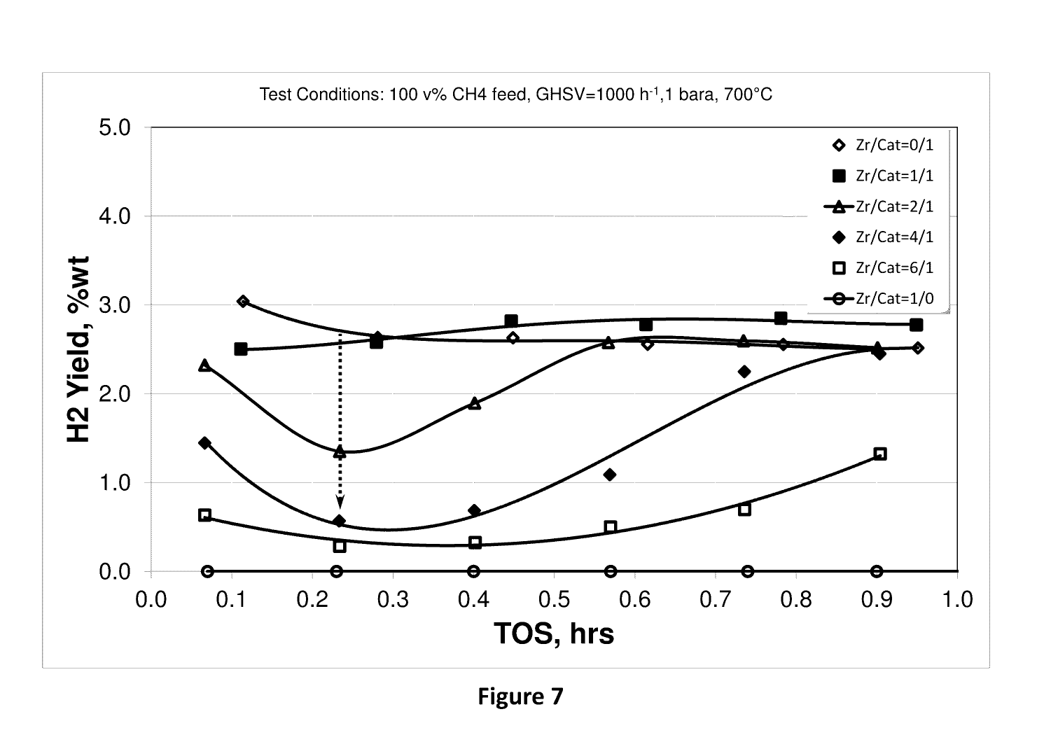

FIG. 7 shows the corresponding hydrogen yields vs. time on stream data obtained for the above samples. The analysis of the data reveals that, the methane aromatization catalyst alone (see curve denoted with Zr:Cat=0/1 and open-diamond shaped line markers) afforded about 2.7% wt of hydrogen yield at TOS of 0.24 hrs. On the other hand, the zirconium acceptor alone was completely inactive for methane aromatization and afforded no appreciable hydrogen yield (see curve denoted as Zr/Cat=1/0 and open circle shaped line markers). In contrast, the zirconium acceptor and methane aromatization catalyst particles mixtures of the present invention with a zirconium acceptor to catalyst particles weight ratios of 1:1 to 6:1 afforded significantly lower hydrogen yields at maximum methane conversion (due to absorption of the produced hydrogen by the zirconium acceptor particles). Specifically, for the mixture with zirconium acceptor: methane aromatization catalyst particles weight ratio of 4:1 (see curve denoted with Zr/Cat=4/1 and solid diamond shaped line markers), the observed hydrogen yield was only about 0.6% wt. In addition, the mixture with Zr:Catalyst particles weight ratio of 6:1 afforded even lower H₂ yield of approximately 0.3% wt (see curve denoted with Zr/Cat=6/1 and open-square shaped line markers). This shows that the 6:1 zirconium acceptor and methane aromatization catalyst particles weight ratio mixture afforded about 90% wt. lower hydrogen yield relative to the methane aromatization catalyst alone. It should be noted that the hydrogen absorbed by the zirconium metal acceptor particles leads to their transformation into zirconium hydride particles. The hydrogen in the zirconium hydride is not permanently bound and/or wasted. The recovery of the hydrogen bound by the zirconium metal acceptor and its utilization and monetization would be desirable from an overall methane aromatization process economics standpoint.

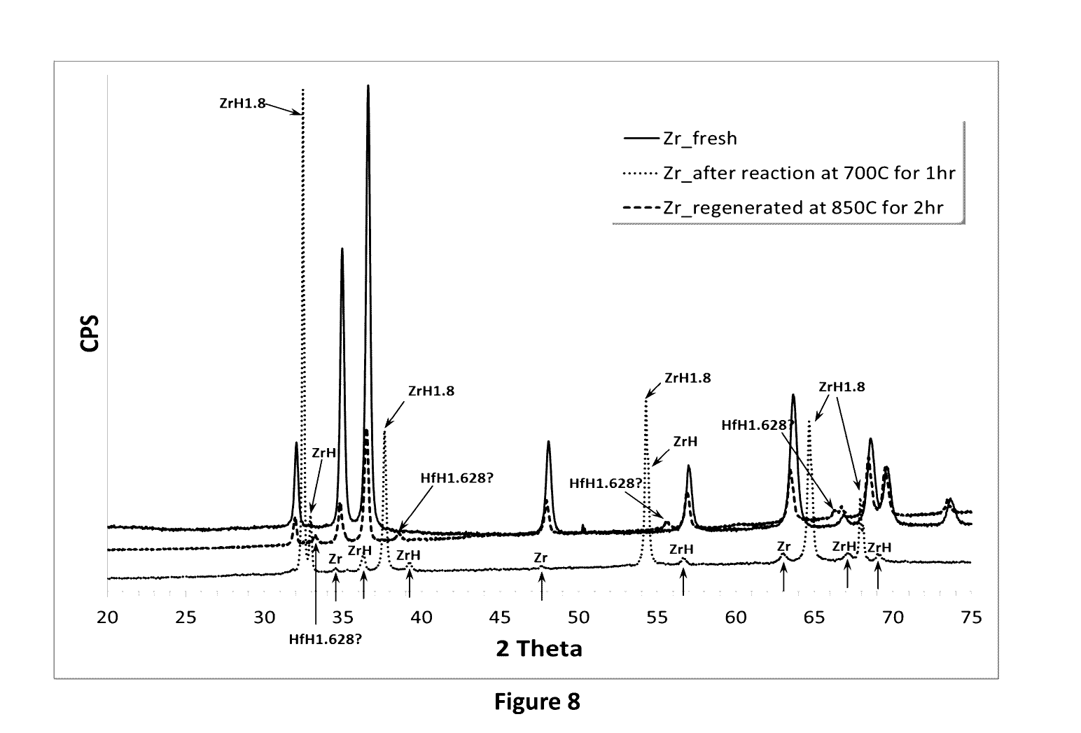

FIG. 8 shows the powder XRD patterns obtained for fresh zirconium particles, spent from the methane aromatization tests zirconium acceptor particles, and spent and regenerated zirconium acceptor particles. The XRD pattern for the fresh zirconium acceptor particles (see the bold solid line curve) shows only XRD reflections characteristic for a zirconium metal phase. No other XRD reflections were observed. The XRD pattern of the spent from the methane aromatization test zirconium acceptor (see dotted line) exhibits XRD reflections typical of ZrHₓ (ZrH_{1.8} and ZrH) crystallographic phases. This could be attributed to the fact that the zirconium hydrogen acceptor underwent partial hydrogenation during the methane aromatization at 700° C. This incompletely saturated zirconium acceptor (ZrHx) following regeneration in inert (Ar) gas at 850° C. exhibited XRD reflections of a pure zirconium metal. No residual reflections characteristic of ZrHₓ were observed following the regeneration. These experiments/data indicates that the zirconium metal acceptor of this invention could effectively be reduced to ZrHₓ in the presence of hydrogen at 700° C. In addition, these data suggest that the so saturated with hydrogen zirconium acceptor (exhibiting ZrHₓ XRD reflections pattern) could be efficiently regenerated during the methane aromatization process by subjecting it to a flow of an inert (Ar) gas at 850° C.

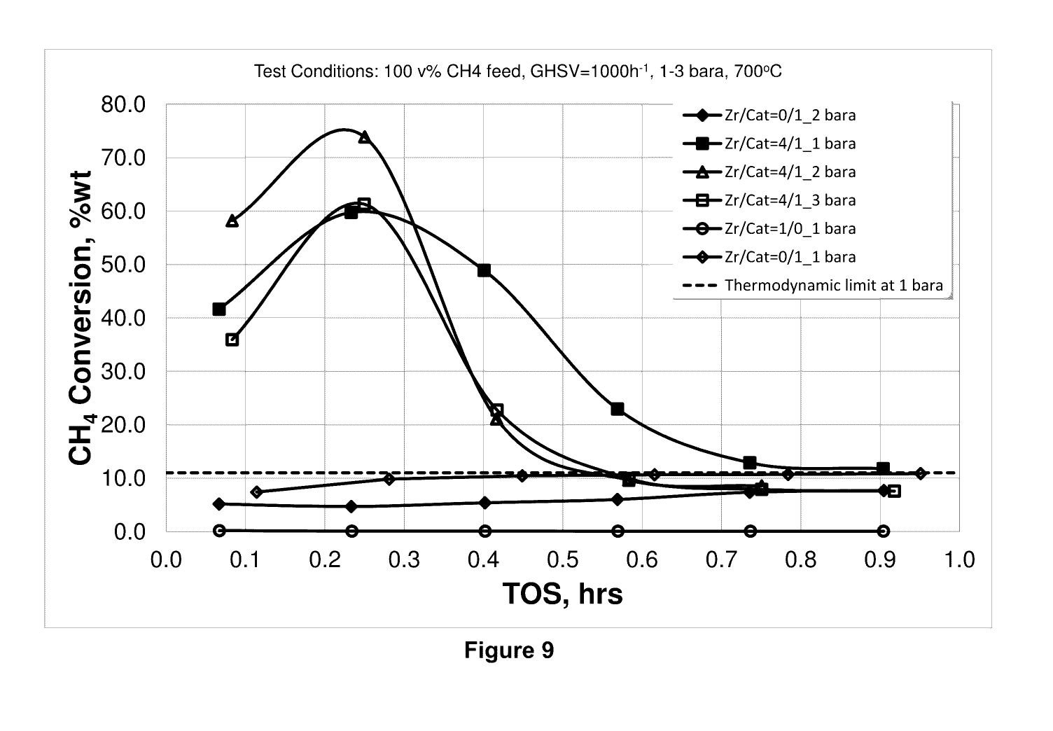

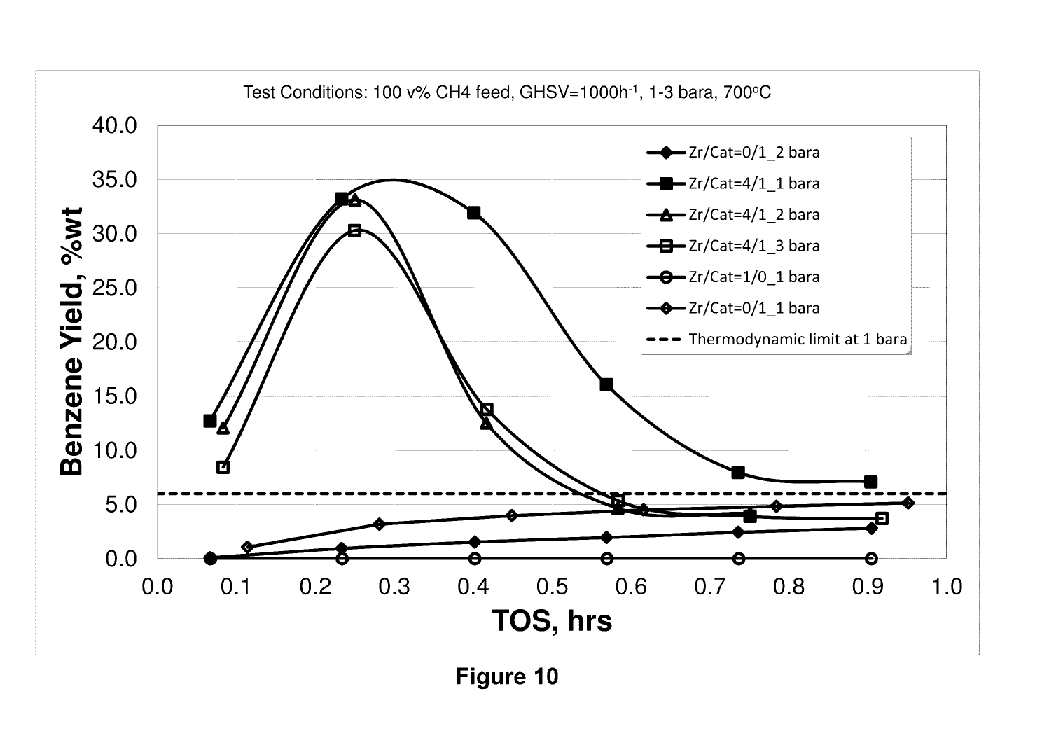

FIGS. 9 and 10 show the effect of operating pressure on the CH₄ conversion and benzene yield vs TOS data obtained for zirconium hydrogen acceptor and methane aromatization catalyst particles mixture of this invention with Zr:Catalyst particles weight ratio of 4:1. For comparison, the Figure also shows CH₄ conversion and benzene yield vs TOS data obtained at different operating pressures for methane aromatization catalyst alone (i.e., according to the prior art without hydrogen acceptor Zr:Catalyst particles weight ratio of 0:1). It is well known that, higher than ambient operating pressures suppress hydrocarbon dehydrogenation reactions rates. The higher the operating pressure the lower the dehydrogenation reaction rates, i.e. lower the corresponding hydrocarbon conversion levels. Since methane aromatization proceeds through formation of dehydrogenated intermediary (C2⁼) hydrocarbon species, the CH₄ conversion (to aromatics) is also suppressed by higher (than ambient) operating pressure. The analysis of the data in FIGS. 9 and 10 shows indeed that, for the methane aromatization catalyst alone (no hydrogen acceptor, prior art) case, the methane conversion and maximum benzene yield at 2 bara (see curves denoted with Zr/Cat=0/1 and solid diamond markers) are significantly lower than (about half of) the CH₄ conversion and benzene yield obtained at 1 bara (see curves denoted with Zr/Cat=0/1 and with open diamond markers). In contrast, for the zirconium hydrogen acceptor and methane aromatization catalyst particles mixture of this invention with Zr:Cat weight ratio of 4:1 (curves with open square, solid square and open triangle line markers) a very significant increase of the CH₄ conversion and corresponding maximum benzene yield beyond the ones dictated by the thermodynamic equilibrium are observed at operating pressures of 1, 2 and 3 bara. Surprisingly, and in contrast to the prior art case, the CH₄ conversion for the acceptor catalyst mixture of this invention remained very similar (from 60-70% wt) within the operating pressure range of 1-3 bara. Also, the benzene yields were not adversely affected and remained very high (in the range of 30-35 wt) at operating pressures ranging from 1-3 bara. This is an unexpected and very beneficial feature of the present invention. The possibility to operate the methane aromatization process at very high CH₄ conversion and benzene yield levels at operating pressures of 2 or 3 bara would allow for significant reduction of the necessary reactor volume, i.e. significant reduction of capital needed for deployment of a commercial methane aromatization process.

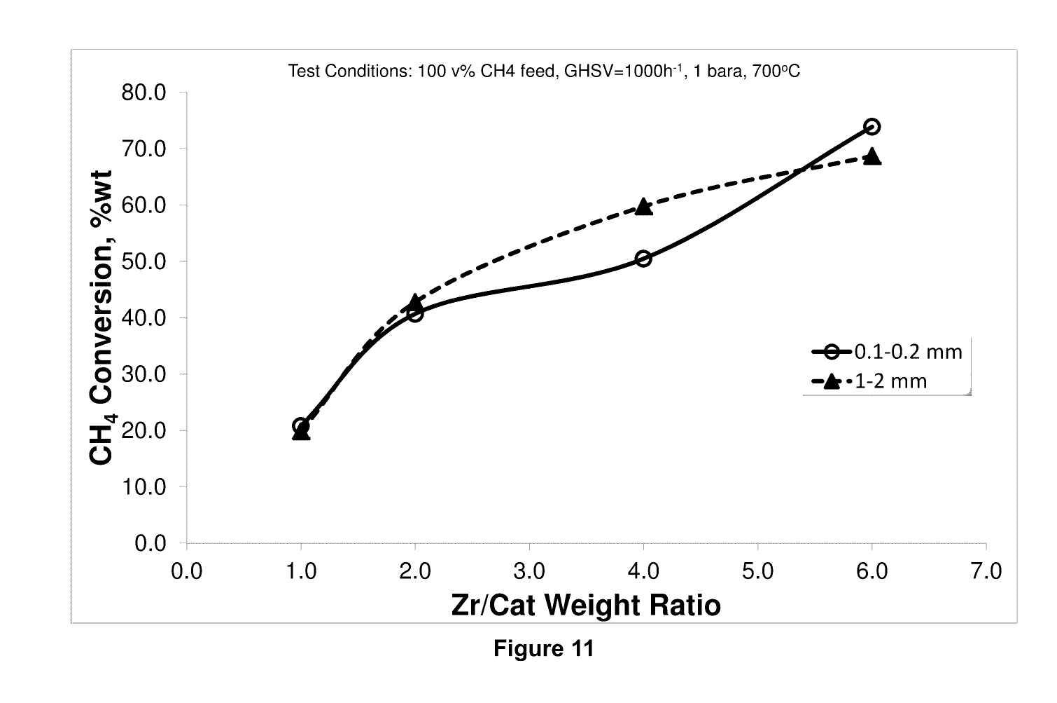

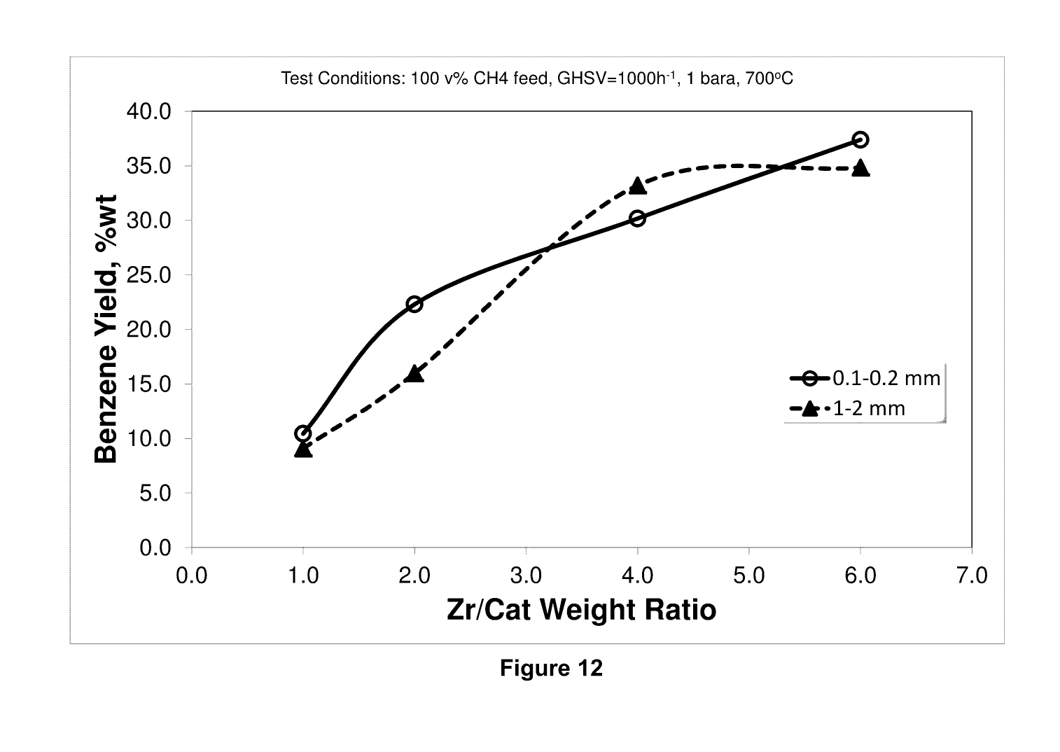

FIGS. 11 and 12 show the effect of the zirconium hydrogen acceptor and methane aromatization catalyst predominant particles size and zirconium acceptor to methane aromatization catalyst particles mixtures weight ratio on CH₄ conversion and benzene yield. Two different particle size mixtures were tested: (i) a zirconium acceptor and catalyst mixture where both, zirconium and catalyst particles were predominantly of the size of 0.1-0.2 mm (curves denoted with open circle line markers) and (ii) a zirconium acceptor and catalyst particles mixture where both zirconium and catalyst particles were predominantly of the size of 1-2 mm (curves denoted with solid triangle line markers). The analysis of the data in the figures shows that the mixtures with both small and large particle sizes afforded in general similar CH₄ conversion and benzene yield levels. The figure also shows that, the trends of the effect of Zr/Catalyst particles weight ratio on CH₄ conversion for small and large zirconium acceptor and catalyst particle sizes are very similar. The figure also shows that, for both small and large acceptor and catalyst particles mixtures the optimal range of zirconium acceptor and methane aromatization catalyst particles mixtures weight ratios remain in the range of greater than or equal to 4:1.

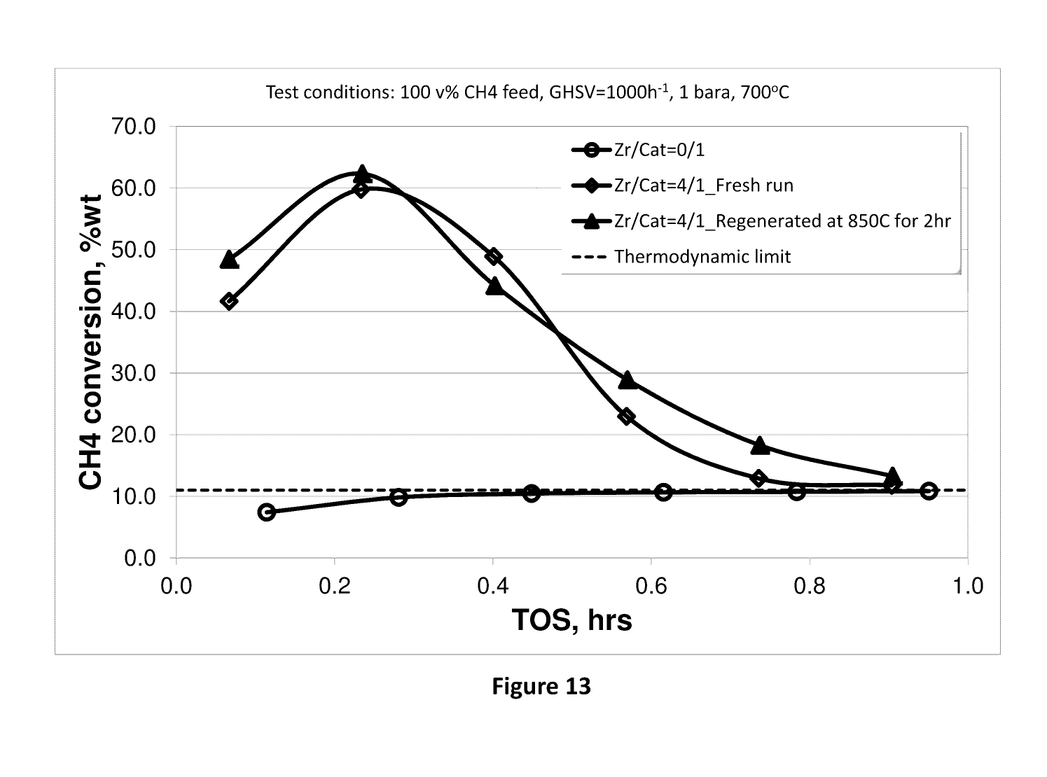

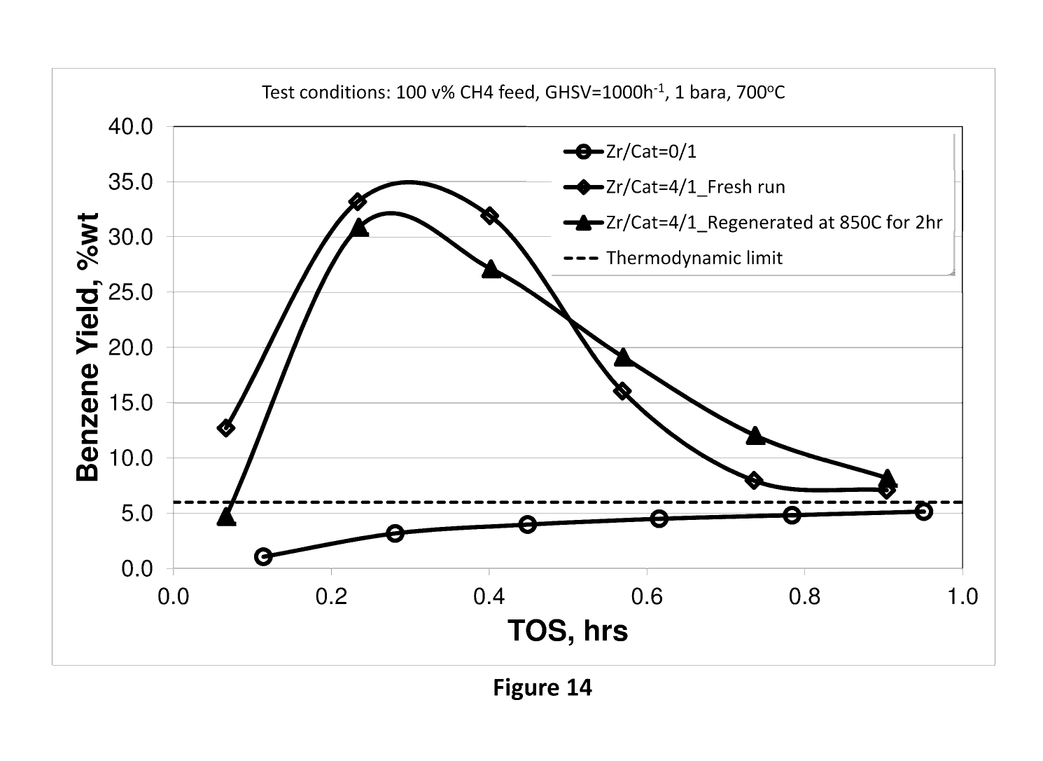

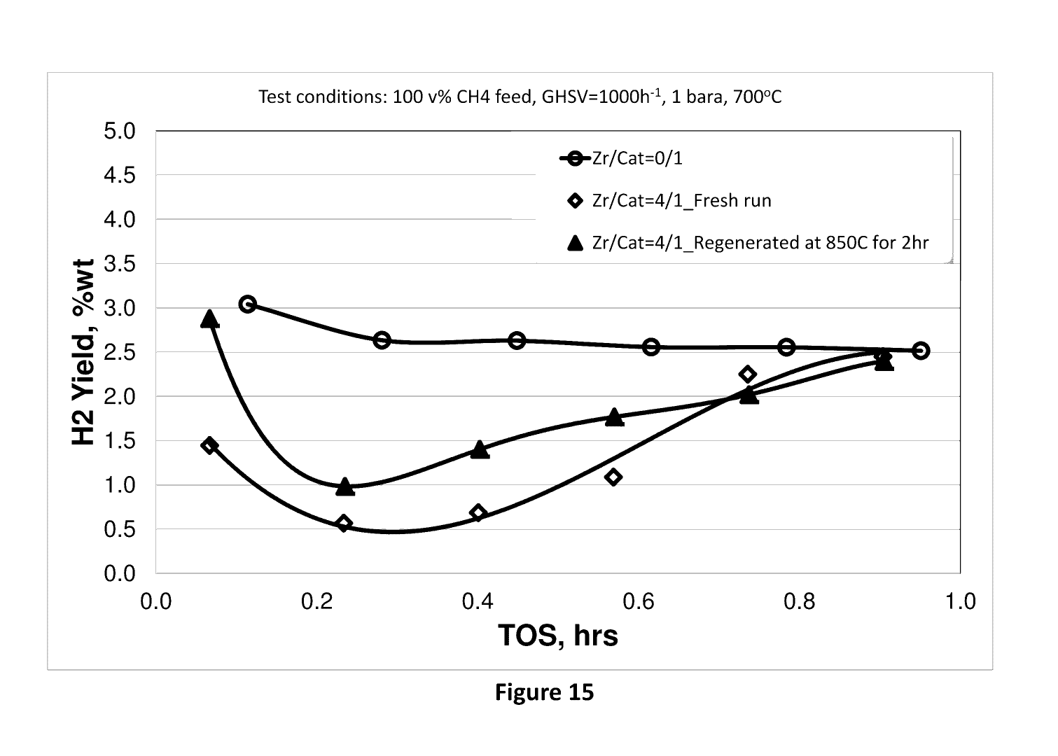

FIGS. 13-15 show the CH₄ conversion, benzene and hydrogen yields versus time on stream data, respectively, gathered for fresh (curves with open diamond line markers) and spent and regenerated (curves with solid triangle line markers) zirconium hydrogen acceptor and methane aromatization catalyst particles mixtures (Zr:Catalyst particles weight ratio of 4:1). The separations of the spent zirconium acceptor particles from the spent methane aromatization catalyst particles, as well as the details of the regeneration procedure for the spent zirconium acceptor, are described in section III P. In order to reliably evaluate the ability to regenerate the spent zirconium acceptor particles, the regenerated spent zirconium acceptor particles were mixed as described above with a fresh methane aromatization catalyst particles to afford the zirconium acceptor: methane aromatization catalyst particles mixture with particles weight ratio of 4:1. This mixture, containing the regenerated zirconium acceptor particles and fresh catalyst particles was then tested under the same test conditions, i.e. by following exactly the same test protocol as the one previously employed for the fresh zirconium acceptor particles and methane aromatization catalyst mixtures with a weight ratio of 4:1. For reference, FIGS. 13-15 show performance data curves for methane aromatization catalyst alone (curves denoted with open circle line markers, no hydrogen acceptor, Zr/Cat=0/1).

The analysis of the data in FIGS. 13-15 shows that the regenerated zirconium acceptor and methane aromatization catalyst particles mixture with particles weight ratio of 4:1 (see curves denoted with Zr/Cat=4/1 and solid triangle shaped line markers) exhibits on average (from the first 2 data points) similar CH₄ conversion and benzene yields relative to the fresh zirconium acceptor and methane aromatization catalyst particles mixture with 4:1 weight ratio (see curves denoted with Zr/Cat=4/1 and open diamond line markers). This demonstrates that the zirconium hydrogen acceptor particles of the presently disclosed subject matter could be regenerated by simply heating them in an inert gas atmosphere at temperature and pressure sufficient to trigger hydrogen desorption. Furthermore, the data suggest that the selected/applied zirconium acceptor regeneration procedure is capable of essentially fully releasing the hydrogen/regenerating the zirconium acceptor particles. Various regeneration gas mediums and operating conditions may be used to optimize the zirconium acceptor particles regeneration procedure and to make it commercially viable.

It is noted that the zirconium acceptor particles could be obtained and commercially utilized as particles of various shapes (spheres, pellets, rods, etc.) and sizes (from 5 microns to 3 cm). The specific shape and size of the zirconium acceptor particles will be dictated by the needs of the particular hydrogen removal application/process. In the specific methane aromatization case, one could expect to obtain even higher methane conversion and aromatics yields per pass by further optimizing: (i) the methane aromatization operating conditions, (ii) the shape and size of zirconium hydrogen acceptor particles, (iii) the homogeneity of the zirconium acceptor and methane aromatization catalyst particles mixing, (iv) the Zr:Cat particles weight ratio, (v) the nature of the zirconium acceptor and methane aromatization catalyst bed/process (fixed, moving or fluidized bed) and (vi) the nature, shape and size of the methane aromatization catalyst particles.

From a methane aromatization process perspective, under the particular fixed-bed operating conditions disclosed herein, the preferred zirconium acceptor particles and methane aromatization catalyst particles mixtures with 4:1 and 6:1 weight ratio afforded peak methane aromatization performance (the highest CH₄ conversion and aromatics yields) for a relatively short period of time on stream (<15 min). Thus, in order to maintain peak performance, a methane aromatization reactor and a regenerator should be selected/configured in such a way to allow for quick insertion and removal of the zirconium hydrogen acceptor and methane aromatization catalyst particles mixture in the reactor zone. This is necessary in order to provide for quick release of hydrogen from the saturated zirconium acceptor and regenerate the zirconium acceptor and methane aromatization catalyst in the regenerator vessel(s) and for quick reinsertion of the rejuvenated zirconium and catalyst particles mixture back into the reaction zone/reactor. The zirconium hydrogen acceptor and methane aromatization catalyst particles mixtures of this invention could be used in a fixed, moving or a fluidized-bed reactor configuration/process.

The foregoing description, for purpose of explanation, has been described with reference to specific embodiments. However, the illustrative discussions above are not intended to be exhaustive or to limit embodiments of the disclosed subject matter to the precise forms disclosed. Many modifications and variations are possible in view of the above teachings. The embodiments were chosen and described in order to explain the principles of embodiments of the disclosed subject matter and their practical applications, to thereby enable others skilled in the art to utilize those embodiments as well as various embodiments with various modifications as may be suited to the particular use contemplated.

What is claimed is:

1. A process for the aromatization of a methane-containing gas stream comprising:
contacting the methane-containing gas stream in a reaction zone of an aromatization reactor comprising an aromatization catalyst and a zirconium hydrogen acceptor under methane-containing gas aromatization conditions to produce a product stream comprising aromatics and hydrogen, wherein at least a portion of the produced hydrogen is bound by the zirconium hydrogen acceptor in the reaction zone and removed from the product stream and the reaction zone, and
wherein the weight ratio of zirconium hydrogen acceptor to the aromatization catalyst is at least 1:1.

2. The process of claim 1, wherein the methane-containing gas stream conversion and corresponding benzene yield per pass are higher than the conversion and yield obtained with the same aromatization catalyst and under the same methane-containing gas aromatization conditions, but in the absence of the weight ratio of zirconium hydrogen acceptor to the aromatization catalyst in the reaction zone of the aromatization reactor.

3. The process of claim 1, wherein the weight ratio of zirconium hydrogen acceptor to the aromatization catalyst is from 1:1 to 10:1.

4. The process of claim 1, wherein the weight ratio of zirconium hydrogen acceptor to the aromatization catalyst is from 2:1 to 6:1.

5. The process of claim 1, wherein the weight ratio of zirconium hydrogen acceptor to the aromatization catalyst is at least 4:1.

6. The process of claim 1, wherein the weight ratio of zirconium hydrogen acceptor to the aromatization catalyst is at least 6:1.

7. The process of claim 1, wherein the obtained conversion of the methane-containing gas stream is at least 35 wt %.

8. The process of claim 1, wherein the obtained benzene yield per pass is at least 15 wt %.

9. The process of claim 1, wherein the methane-containing gas stream further comprises at least one compound selected from the group consisting of ethane, propane, butane, and carbon dioxide.

10. The process of claim 1, wherein the aromatization reactor is a fixed bed reactor.

11. The process of claim 1, wherein the zirconium hydrogen acceptor comprises one or more metals.

12. The process of claim 1, wherein the methane aromatization conditions comprise a temperature in the range of from 500° C. to 900° C.

13. The process of claim 1, wherein the methane aromatization conditions comprise a temperature in the range of from 600° C. to 800° C.

14. The process of claim 1, further comprising continuously regenerating the catalyst to remove coke formed during the reaction and continuously regenerating the zirconium hydrogen acceptor by releasing the hydrogen under regeneration conditions.

15. The process of claim 14, wherein the catalyst and hydrogen acceptor are regenerated in a single regeneration vessel.

16. The process of claim 14, wherein the catalyst and hydrogen acceptor are regenerated in separate vessels.

17. The process of claim 1, wherein the catalyst and hydrogen acceptor are each regenerated under different regeneration conditions.

18. The process of claim 14, wherein the hydrogen released from the hydrogen acceptor during regeneration of the hydrogen acceptor is used for catalyst regeneration.

19. The process of claim 18, wherein supplemental hydrogen is supplied from an external source in order to properly complete the catalyst regeneration.

20. The process of claim 14, wherein the zirconium hydrogen acceptor regeneration is accomplished under regeneration conditions including: feed rate, temperature and pressure that are substantially different from the aromatization conditions.

21. The process of claim 14, wherein the zirconium acceptor regeneration conditions include a regeneration gas GHSV of from 500-10,000 h-1, a temperature of from 700-950° C. and pressure of from 0.5-4 bara.
